SEQUENCE LISTING (12 sequences)

SEQ ID NO: 1 - DNA, 23 bp, Homo sapiens
aacattcattgctgtcggtgggt

SEQ ID NO: 2 - DNA, 22 bp, Homo sapiens
tgcgggtgctcgcttcggcagc

SEQ ID NO: 3 - DNA, 24 bp, Homo sapiens
aatctcgaggaaccacagcttcct

SEQ ID NO: 4 - DNA, 24 bp, Homo sapiens
tccgaattcactccatgttagaac

SEQ ID NO: 5 - DNA, 28 bp, Homo sapiens
ggtcacaatcagggaaagggaaagtcgg

SEQ ID NO: 6 - DNA, 28 bp, Homo sapiens
ccgactttccctttccctgattgtgacc

SEQ ID NO: 7 - DNA, 26 bp, Homo sapiens
ggtaacgcgtcattatcttgtcactg

SEQ ID NO: 8 - DNA, 26 bp, Homo sapiens
gggcaagcttctatttaactctgacc

SEQ ID NO: 9 - DNA, 25 bp, Homo sapiens
attaactttgcccgtgactctgttc

SEQ ID NO: 10 - DNA, 25 bp, Homo sapiens
gaacagagtcacgggcaaagttaat

SEQ ID NO: 11 - DNA, 27 bp, Homo sapiens
gttagaccgttgcccatgatataaaag

SEQ ID NO: 12 - DNA, 27 bp, Homo sapiens
cttttatatcatgggcaacggtctaac

(19) United States
(12) Patent Application Publication
(10) Pub. No.: US 2021/0275521 A1
(43) Pub. Date: Sep. 9, 2021

(54) OXABICYCLOHEPTANES FOR TREATMENT OF SECONDARY ACUTE MYELOID LEUKEMIA

(71) Applicant: Lixte Biotechnology, Inc., East Setauket, NY (US)

(72) Inventor: John S. KOVACH, East Setauket, NY (US)

(21) Appl. No.: 16/770,136

(22) PCT Filed: Dec. 5, 2018
(86) PCT No.: PCT/US2018/063980

§ 371 Date: Jun. 5, 2020

Related U.S. Application Data
(60) Provisional application No. 62/594,772, filed on Dec. 5, 2017.

(51) Int. Cl. A61K 31/496; A61K 31/704; A61P 35/02

(52) U.S. Cl. CPC A61K 31/496; A61P 35/02; A61K 31/704

(57) ABSTRACT

The present invention relates to compounds and methods useful for treating secondary acute myeloid leukemia (sAML).

### CROSS REFERENCE TO RELATED APPLICATIONS

This application is a § 371 National Stage of PCT International Application No. PCT/US2018/063980 which claims the benefit of U.S. Provisional patent application Ser. No. 62/594,772, filed Dec. 5, 2017, the entirety of each of which is hereby incorporated herein by reference.

### SEQUENCE LISTING

This application contains a Sequence Listing which has been submitted in ASCII format via EFS and is hereby incorporated by reference. The ASCII copy, created on Apr. 9, 2021 is named Lixte_034US1_ST25.txt and is 2,134 bytes in size.

### BACKGROUND OF THE INVENTION

Myelodysplastic syndromes (MDS) are a group of hematological disorders characterized by hematopoietic progenitor cells with dysplastic cell morphology, ineffective hematopoiesis, and potential for clonal evolution¹. MDS represent the most common cause of acquired bone marrow failure in adults, and up to 30% of patients progress to secondary acute myeloid leukemia (sAML)²⁻⁵. Evolution to late stage MDS involves upregulation of anti-apoptotic proteins such as Bcl-2, and downregulation of pro-apoptotic proteins such as Fas and Myc⁶⁻⁸. Transformation to sAML has been linked to inactivation of tumor suppressive genes such as p53 and p15^{Ink4b 9,10}. Collectively these changes result in a diminished ability for cell cycle control, and contribute to the aggressive phenotype and chemoresistant behavior typified by sAML⁵. More effective therapeutic strategies are urgently needed to help patients afflicted with this grave condition.

Protein phosphatase 2A (PP2A) is a highly conserved dual-specificity phosphatase that plays a pivotal role in regulating cell cycle protein activity and inhibition of apoptosis through direct interaction with serine/threonine phosphorylation switches¹¹⁻¹³. It is often seen with elevated activity and/or expression in neoplastic cells where it functions as a positive regulator of cell growth and survival¹⁴⁻¹⁶. PP2A promotes resistance to apoptosis through direct dephosphorylation of Bcl-2¹⁷, and through dephosphorylative activation of the inhibitory kinase of caspase-2, CaMKII¹⁸. PP2A is a positive regulator of Ras/Raf/MEK/ERK signaling, an anti-apoptotic pathway well characterized in states of malignant transformation¹⁹⁻²³. Targeted inhibition of PP2A in p53 overexpressing HeLa cells has been shown to induce cell cycle arrest at least partially through increased levels of the Cdk5 activator, p25. Upregulated Cdk5 in turn facilitates Bax translocation into the mitochondrial membrane to promote apoptosis²⁴. Similarly, PP2A inhibition of T leukemia cells has been demonstrated to result in caspase-dependent apoptosis through p38 MAPK activation and loss of mitochondrial transmembrane potential²⁵. PP2A inhibition in human myeloid cell lines induces cell cycle arrest and apoptosis through increased degradation of Bd-2 mRNA, although the direct mechanism of transcript destabilization has not yet been seen²⁶⁻²⁹. PP2A inhibition has shown promise in the treatment multiple tumor types including glioma, sarcoma, pancreatic cancer and del(5q) MDS³⁰⁻³³. Hence, targeting PP2A may be a potential strategy in sAML chemotherapy.

Pharmacologic inhibition of PP2A has generally been studied using a variety of naturally produced, but toxic molecules. Okadaic acid is a PP1 and PP2A inhibitor produced by dinoflagellates presumably as a cytotoxic self-defense agent³⁴. Although it exhibits potent apoptotic effects in many human cancer cell lines³⁵⁻³⁷, its neuro-toxic and enterogenic effects limit its us^{38,39}. Cantharidin is an odorless organic chemical secreted by the blister beetle used for more than 2000 years in traditional Chinese medicine to treat a variety of disorders including MCV infections and warts⁴⁰. Cantharadin is a selective PP2A inhibitor that induces cell-cycle arrest and apoptosis in a variety of cancer subtypes such as breast, colon, pancreatic, hepatocellular, and bladder carcinoma⁴¹⁻⁴⁹. Nevertheless, cantharidin is associated with severe side effects due to high gastrointestinal and renal toxicity^{50,51}. Researchers have recently focused on LB100, a synthetic cantharidin with specific PP2A inhibitory activity that does not appear to exhibit significant systemic toxicity^{32,52,53}. LB100 has shown promising anti-neoplastic activity as a solo chemotherapy agent, and also as a radio- and chemotherapy sensitizer against glioblastoma, pheo-chromocytoma, breast cancer, nasopharyngeal cancer, hepatocellular carcinoma, pancreatic cancer, and ovarian cancer^{31,33,52-58}. It has also shown synergistic cytotoxic effects with doxorubicin to inhibit progression of stem cell-derived aggressive sarcoma³². As such, it is currently in Phase I clinical trials as a potential treatment against progressive and metastatic solid tumors⁵⁹, with another phase I clinical trial planned for the treatment of low-risk MDS resistant to lenalidomide³⁰. However, LB100 has not yet been studied in models of sAML, and its mechanism of chemosensitization has not been directly elucidated.

Patients with secondary acute myeloid leukemia (sAML) arising from myelodysplastic syndromes have a poor prognosis marked by an increased resistance to chemotherapy. An urgent need exists for adjuvant treatments that can enhance or replace current therapeutic options. Here the potential of LB100, a small-molecule protein phosphatase 2 A (PP2A) inhibitor, as a monotherapy and chemosensitizing agent for sAML using an in-vitro and in-vivo approach is demonstrated.

### SUMMARY OF THE INVENTION

The present invention provides a method of treating sAML in a subject comprising administering a PP2A inhibitor so as to thereby treat sAML.

The present invention also provides a method of enhancing cytotoxicity of an anti-cancer agent in a subject afflicted with sAML comprising administering to the subject a PP2A inhibitor so as to thereby enhance cytotoxicity of the anti-cancer agent.

The present invention also provides a method of enhancing cytotoxicity of an anti-cancer agent in a subject afflicted with sAML via upregulation of miR-181b-1 comprising administering to the subject a PP2A inhibitor so as to thereby enhance cytotoxicity of the anti-cancer agent.

The present invention also provides a method of treating sAML in a subject comprising administering a PP2A inhibitor in combination with an anti-cancer agent so as to thereby treat sAML, wherein the amounts when taken together are effective to treat the subject.

The present invention also provides a method of enhancing cytotoxicity of daunorubicin in a subject afflicted with sAML comprising administering to the subject a PP2A inhibitor so as to thereby enhance cytotoxicity of the daunorubicin.

The present invention also provides a method of enhancing cytotoxicity of daunorubicin in a subject afflicted with sAML via upregulation of miR-181b-1 comprising administering to the subject a PP2A inhibitor so as to thereby enhance cytotoxicity of the daunorubicin.

The present invention also provides a method of treating sAML in a subject comprising administering a PP2A inhibitor in combination with daunorubicin so as to thereby treat sAML, wherein the amounts when taken together are effective to treat the subject.

### BRIEF DESCRIPTION OF THE DRAWINGS

FIG. 1 depicts a graph showing cell proliferation with LB100 in multiple leukemia cell lines in a dose dependent manner.

FIG. 2 depicts a graph showing SKM-1 colony formation rate following LB100 treatment in a concentration dependent fashion after 7 days of culture in methylcellulose medium.

FIG. 3 depicts images of colony formation following LB100 treatment in a concentration dependent fashion after 7 days of culture in methylcellulose medium.

FIG. 4 depicts a graph showing PP2A activity with increasing concentrations of LB100 in SKM-1 cells after 6 hours of LB100 treatment

FIG. 5 depicts PP2A isoform levels after 5 μM LB100 treatment for 12 h. Statistically significant differences are marked by an asterisk (*P<0.05; **P<0.01, ***PC 0.001).

FIG. 6 depicts flow cytometry analysis in various phases of the cell cycle after 5RM LB100 treatment for 0, 6 and 12 h.

FIG. 7 depicts a graph showing percentage of SKM-1 cells in various phases of the cell cycle after 5RM LB100 treatment for 0, 6 and 12 h.

FIG. 8 depicts time-dependent decrease in G2/M regulatory proteins after 5 μM LB100 treatment in SKM-1 cells.

FIG. 9. depicts flow cytometry analysis of SKM-1 cells stained with annexin V and propidium iodide.

FIG. 10 depicts a dose dependent increase in cleaved caspase 3 and PARP after 24h exposure to LB100 in SKM-1 cells.

FIG. 11 depicts fluorescent microscopy analysis of Hoechst-stained SKM-1 cells after LB100 treatment at varying doses.

FIG. 12 depicts flow cytometry analysis after 24 h of LB100 treatment (10 μM) or control, in the presence or absence of z-VAD-FMK. Statistically significant differences are marked by an asterisk (*P<0.05; **Pc0.01, ***P<0.001).

FIG. 13 depicts a graph showing cytotoxic activity of daunorubicin in combination with LB100 in SKM-1 cells. Statistically significant differences are marked by an asterisk (*P<0.05; **P<0.01, ***PC 0.001).

FIG. 14 depicts a graph showing cytotoxic activity of daunorubicin in combination with LB100 in a primary sAML patient sample. Statistically significant differences are marked by an asterisk (*P<0.05; **P<0.01, ***PC 0.001).

FIG. 15 depicts a graph showing cytotoxic activity of daunorubicin in combination with LB100 in a primary sAML patient sample. Statistically significant differences are marked by an asterisk (*P<0.05; **P<0.01, ***PC 0.001).

FIG. 16 depicts a graph showing cytotoxic activity of daunorubicin in combination with LB100 in a primary sAML patient sample. Statistically significant differences are marked by an asterisk (*P<0.05; **P<0.01, ***PC 0.001).

FIG. 17 depicts tumor volume of mice tumors treated with daunorubicin in combination with LB100.

FIG. 18 depicts a graph showing tumor volume of mice tumors treated with daunorubicin in combination with LB100.

FIG. 19 depicts a graph showing overall survival of mice treated with daunorubicin in combination with LB100. Statistically significant differences are marked by an asterisk (*P<0.05; ***P<0.001).

FIG. 20 depicts a graph showing expression of miR-181b-1 in SKM-1 cells exposed to LB100.

FIG. 21 depicts a western blot of SKM-1 cells after 5 μM of LB100 treatment for 0, 3, 6 and 12 h.

FIG. 22 depicts a predicted miR-181b-1 sequence complementarity to the 3′ untranslated region of Bd-2 mRNA.

FIG. 23 depicts SKM-1 xenograft histology after LB100 treatment. Statistically significant differences are marked by an asterisk (*P<0.05).

FIG. 24 depicts a graph showing relative luciferase activity in 293T cells when pMIR-REPORT-Bcl-2-3′UTR was coinfected with miR-181b-1 retrovirus, but not with normal control (NC) or mut-miR-181b-1.

FIG. 25 depicts GFP analysis of SKM-1 cells infected with miR-181b-1 retrovirus.

FIG. 26 depicts a graph showing expression of miR-181b-1 in SKM-1 cells.

FIG. 27 depicts a graph showing expression of Bcl-2 mRNA in SKM-1 cells.

FIG. 28 depicts induced expression of cleaved caspase 3.

FIG. 29 depicts a graph showing reversal of LB100 (2.511, M) induced SKM-1 cell death after administration of anti-miRNA targeting miR-181b-1 (20 nM).

FIG. 30 depicts a graph showing overexpression of miR-181b-1 and cytotoxic activity of daunorubicin in sAML cells. Statistically significant differences are marked by an asterisk (*P<0.05; **P<0.01, ***P<0.001).

### DETAILED DESCRIPTION OF CERTAIN EMBODIMENTS OF THE INVENTION

### 1. General Description of Certain Embodiments of the Invention

In one aspect, the present invention provides a method of treating sAML in a subject comprising administering an effective amount of a PP2A inhibitor to the subject so as to thereby treat sAML.

The present invention also provides a method of enhancing cytotoxicity of an anti-cancer agent in a subject afflicted with sAML comprising administering to the subject an effective amount of PP2A inhibitor so as to thereby enhance cytotoxicity of the anti-cancer agent.

The present invention also provides a method of enhancing cytotoxicity of an anti-cancer agent in a subject afflicted with sAML via upregulation of miR-181b-1 comprising administering to the subject an effective amount of a PP2A inhibitor so as to thereby enhance cytotoxicity of the anti-cancer agent.

The present invention also provides a method of treating sAML in a subject comprising administering an effective amount of a PP2A inhibitor in combination with an anti-cancer agent so as to thereby treat sAML, wherein the amounts when taken together are effective to treat the subject.

The present invention also provides a method of enhancing cytotoxicity of daunorubicin in a subject afflicted with sAML comprising administering to the subject an effective amount of a PP2A inhibitor so as to thereby enhance cytotoxicity of the daunorubicin.

The present invention also provides a method of enhancing cytotoxicity of daunorubicin in a subject afflicted with sAML via upregulation of miR-181b-1 comprising administering to the subject an effective amount of a PP2A inhibitor so as to thereby enhance cytotoxicity of the daunorubicin.

The present invention also provides a method of treating sAML in a subject comprising administering an effective amount of a PP2A inhibitor in combination with daunorubicin so as to thereby treat sAML, wherein the amounts when taken together are effective to treat the subject.

In some embodiments, the above method further comprises administering an anti-cancer agent concurrently with, prior to, or after the PP2A inhibitor.

In some embodiments of any of the above methods, the amount of PP2A inhibitor and the amount of the anti-cancer agent are each periodically administered to the subject.

In some embodiments of any of the above methods, the amount of PP2A inhibitor and the amount of the anti-cancer agent are administered simultaneously, separately or sequentially.

In some embodiments of any of the above methods, the amount of PP2A inhibitor and the amount of the anti-cancer agent when administered together is more effective to treat the subject than when each agent at the same amount is administered alone.

In some embodiments of any of the above methods, the amount of PP2A inhibitor and the amount of the anti-cancer agent when taken together is effective to reduce a clinical symptom of the cancer in the subject.

In some embodiments of any of the above methods, the PP2A inhibitor enhances the chemotherapeutic effect of the anti-cancer agent.

In some embodiments of any of the above methods, the anti-cancer agent is an daunorubicin.

In some embodiments of any of the above methods, the PP2A inhibitor is a compound having the structure

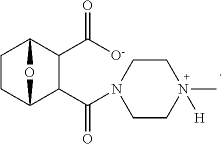

In some embodiments of any of the above methods, the PP2A inhibitor has the structure:

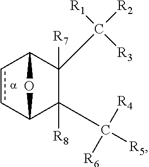

wherein

bond α is present or absent;

R₁ and R₂ together are ═O;

R₃ is OH, O⁻, OR₉, O(CH₂)₁₋₆R₉, SH, S⁻, or SR₉,

wherein R₉ is H, alkyl, alkenyl, alkynyl or aryl;

R₄ is

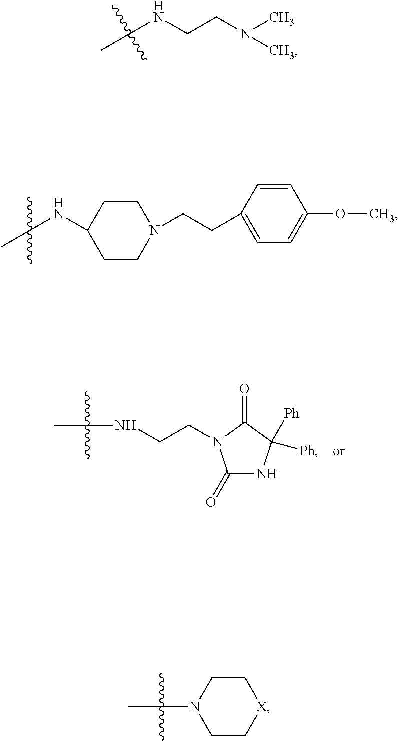

where X is O, S, NR₁₀, N⁺HR₁₀ or N⁺R₁₀R₁₀,

where each R₁₀ is independently H, alkyl, alkenyl, alkynyl, aryl,

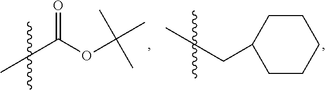

CH₂CN, —CH₂CO₂R₁₁, or —CH₂COR₁₁,
wherein each R₁₁ is independently H, alkyl, alkenyl or alkynyl;

R₅ and R₆ taken together are ═O;

R₇ and R₈ are each H,

or a salt, zwitterion, or ester thereof.

In some embodiments of any of the above methods, the compound has the structure:

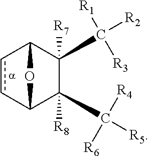

In some embodiments, bond α in the compound is present.

In some embodiments, bond α in the compound is absent.

In some embodiments, R₃ is OH, O″, or OR₉, wherein R₉ is alkyl, alkenyl, alkynyl or aryl;

R₄ is

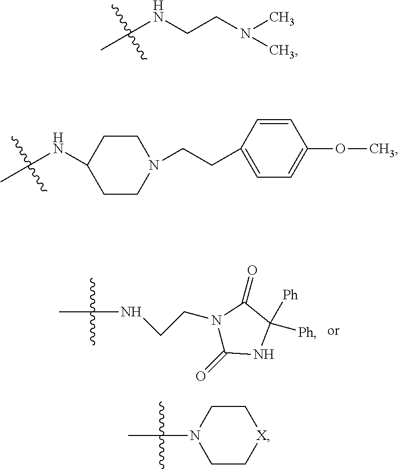

where X is O, S, NR₁₀, N⁺HR₁₀ or N⁺R₁₀R₁₀,

where each R₁₀ is independently H, alkyl, alkenyl, alkynyl, aryl,

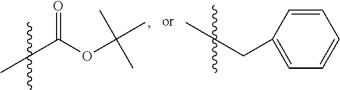

In some embodiments, R₃ is OH, O— or OR₉, where R₉ is H, methyl, ethyl or phenyl.

In some embodiments, R₃ is OH, O⁻ or OR₉, wherein R₉ is methyl.

In some embodiments, R₄ is

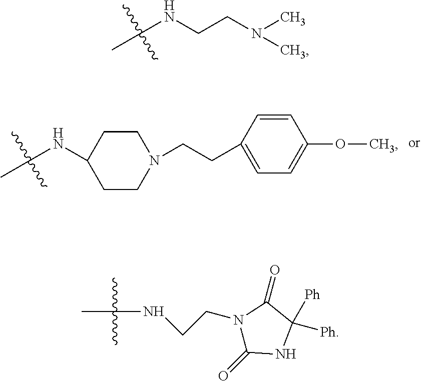

In some embodiments, R₄ is

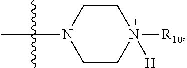

wherein R₁₀ is H, alkyl, alkenyl, alkynyl, aryl, or

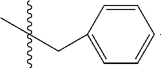 [SMILES: *Cc1ccccc1 ; "."]

In some embodiments, R₄ is

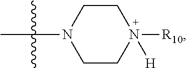

wherein R₁₀ is —H, —CH₃, —CH₂CH₃, or

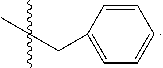 [SMILES: *Cc1ccccc1 ; "."]

In some embodiments, R₄ is

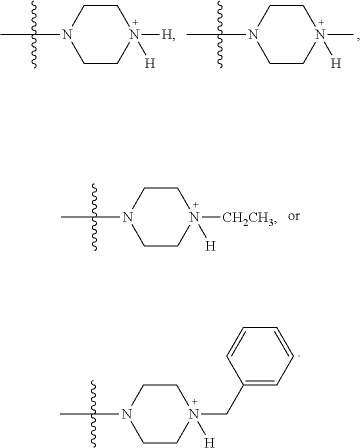

In some embodiments, R₄ is

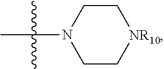

wherein R₁₀ is H, alkyl, alkenyl, alkynyl, aryl,

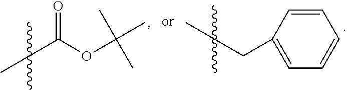

In some embodiments, R₄ is

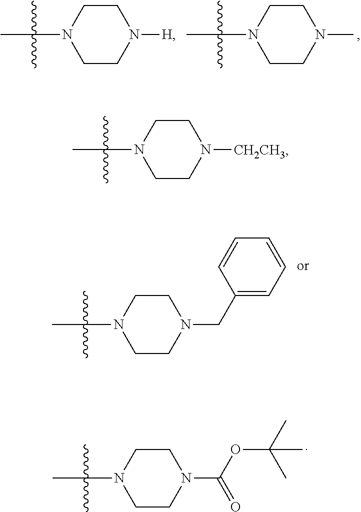

In some embodiments, R₄ is

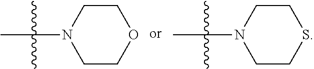 [SMILES: *N1CCOCC1 ; "or" ; *N1CCSCC1]

In some embodiments of any of the above methods, the compound has the structure

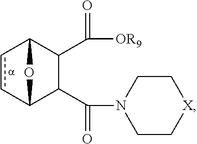

wherein

bond α is present or absent;

R₉ is present or absent and when present is H, alkyl, alkenyl, alkynyl or phenyl; and

X is O, NR₁₀, NH⁺R₁₀ or N⁺R₁₀R₁₀,

where each R₁₀ is independently H, alkyl, substituted alkyl, alkenyl, substituted alkenyl, alkynyl, substituted alkynyl, aryl,

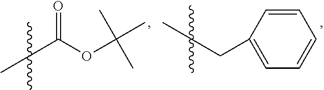

—CH₂CN, —CH₂CO₂R₁₂, or —CH₂COR₁₂, where R₁₂ is H or alkyl,

or a salt, zwitterion or ester thereof.

In some embodiments of any of the above methods, the compound has the structure

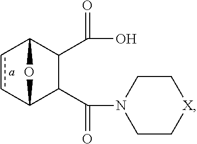

wherein

bond α is present or absent;

### X is O or NR₁₀,

where each R₁₀ is independently H, alkyl, substituted alkyl, alkenyl, substituted alkenyl, alkynyl, substituted alkynyl, aryl,

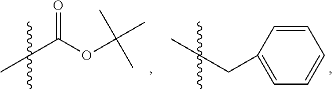

—CH₂CN, —CH₂CO₂R₁₂, or —CH₂COR₁₂, where R₁₂ is H or alkyl,

or a salt, zwitterion or ester thereof.

In some embodiments of any of the above methods, the compound has the structure

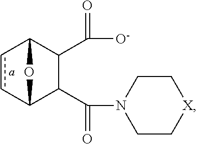

wherein

bond α is present or absent;

X is O or NH⁺R₁₀,

where R₁₀ is H, alkyl, substituted alkyl, alkenyl, substituted alkenyl, alkynyl, substituted alkynyl, aryl,

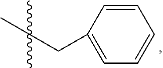 [SMILES: *Cc1ccccc1]

—CH₂CN, —CH₂CO₂R₁₂, or —CH₂COR₁₂, where R₁₂ is H or alkyl, or a salt, zwitterion or ester thereof.

In some embodiments of any of the above methods, the compound has the structure

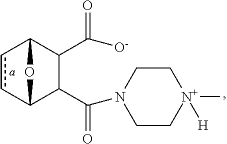

or a salt or ester thereof.

In some embodiments of any of the above methods, the compound has the structure

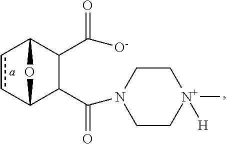

or a salt or ester thereof.

In some embodiments of any of the above methods, the compound has the structure:

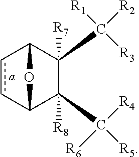

In some embodiments, bond α in the compound is present.

In some embodiments, bond α in the compound is absent.

In some embodiments, R₃ is OH, O⁻, or OR₉, wherein R₉ is alkyl, alkenyl, alkynyl or aryl;

R₄ is

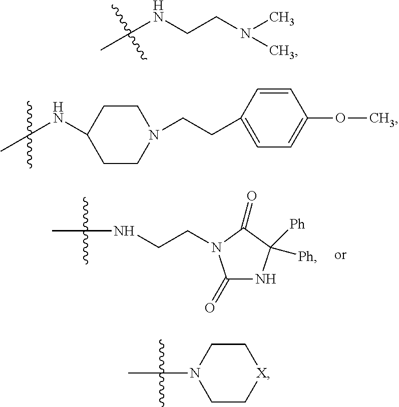

where X is O, S, NR₁₀, N⁺HR₁₀ or N⁺R₁₀R₁₀, where each R₁₀ is independently H, alkyl, alkenyl, alkynyl, aryl,

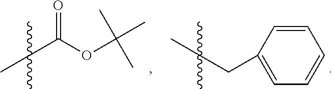

In some embodiments, R₃ is OH, O⁻ or OR₉, where R₉ is H, methyl, ethyl or phenyl.

In some embodiments, R₃ is OH, O⁻ or OR₉, wherein R₉ is methyl.

In some embodiments, R₄ is

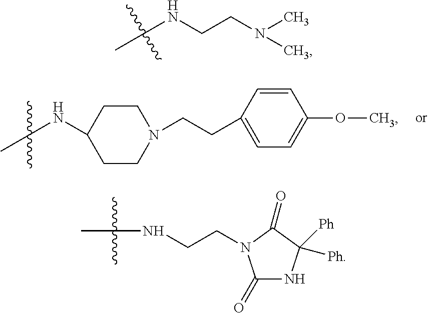

In some embodiments, R₄ is

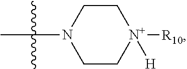

wherein R₁₀ is H, alkyl, alkenyl, alkynyl, aryl, or

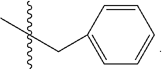 [SMILES: *Cc1ccccc1]

In some embodiments, R₄ is

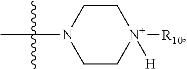

wherein R₁₀ is —H, —CH₃, —CH₂CH₃, or

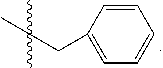 [SMILES: *Cc1ccccc1]

In some embodiments, R₄ is

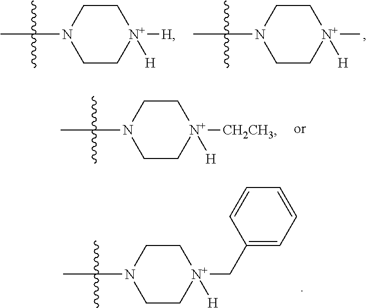

In some embodiments, R₄ is

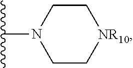

wherein R₁₀ is H, alkyl, alkenyl, alkynyl, aryl,

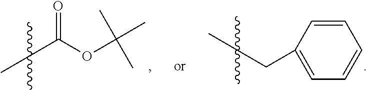

In some embodiments, R₄ is

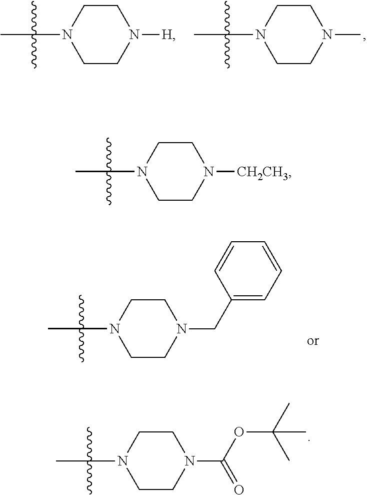

In some embodiments, R₄ is

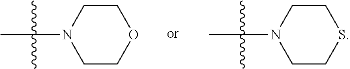 [SMILES: *N1CCOCC1 ; *N1CCSCC1]

In some embodiments of any of the above methods, the compound has the structure:

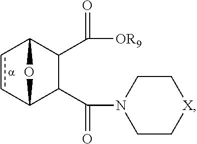

wherein

bond α is present or absent;
R₉ is present or absent and when present is H, alkyl, alkenyl, alkynyl or phenyl; and
X is O, NR₁₀, NE⁺R₁₀ or N⁺R₁₀R₁₀, where each R₁₀ is independently H, alkyl, substituted alkyl, alkenyl, substituted alkenyl, alkynyl, substituted alkynyl, aryl,

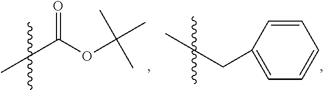

—CH₂CN, —CH₂CO₂R₁₂, or —CH₂COR₁₂, where R₁₂ is H or alkyl,

or a salt, zwitterion or ester thereof.

In some embodiments of any of the above methods, the compound has the structure:

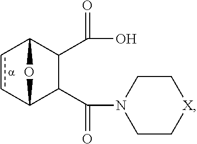

wherein

bond α is present or absent;

### X is O or NR₁₀,

where each R₁₀ is independently H, alkyl, substituted alkyl, alkenyl, substituted alkenyl, alkynyl, substituted alkynyl, aryl,

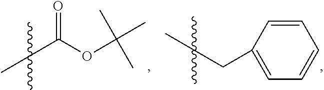

—CH₂CN, —CH₂CO₂R₁₂, or —CH₂COR₁₂, where R₁₂ is H or alkyl,
or a salt, zwitterion or ester thereof.

In some embodiments of any of the above methods, the compound has the structure:

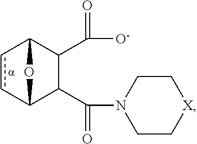

wherein

bond α is present or absent;

X is O or NH⁺R₁₀,

where R₁₀ is H, alkyl, substituted alkyl, alkenyl, substituted alkenyl, alkynyl, substituted alkynyl, aryl,

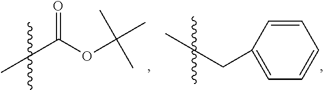

—CH₂CN, —CH₂CO₂R₁₂, or —CH₂COR₁₂, where R₁₂ is H or alkyl,

or a salt, zwitterion or ester thereof.

In some embodiments of any of the above methods, the compound has the structure:

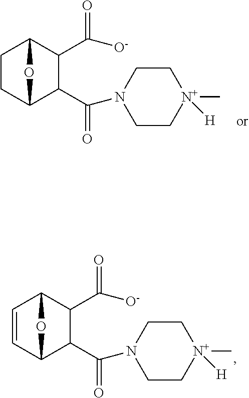 [SMILES: C[NH+]1CCN(C(=O)C2C(C(=O)[O-])[C@H]3CC[C@@H]2O3)CC1 ; C[NH+]1CCN(C(=O)C2C(C(=O)[O-])[C@H]3C=C[C@@H]2O3)CC1]

or a salt or ester thereof.

In one embodiment, the compound of the method has the structure:

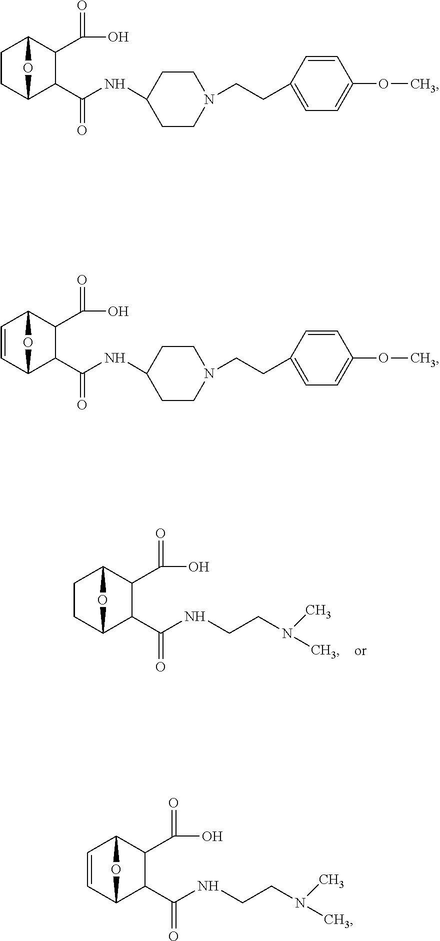 [SMILES: COc1ccc(CCN2CCC(NC(=O)C3C(C(=O)O)[C@H]4CC[C@@H]3O4)CC2)cc1 ; COc1ccc(CCN2CCC(NC(=O)C3C(C(=O)O)[C@H]4C=C[C@@H]3O4)CC2)cc1 ; CN(C)CCNC(=O)C1C(C(=O)O)[C@H]2CC[C@@H]1O2 ; CN(C)CCNC(=O)C1C(C(=O)O)[C@H]2C=C[C@@H]1O2]

or a salt, zwitterion, or ester thereof.

In one embodiment, the compound of the method has the structure:

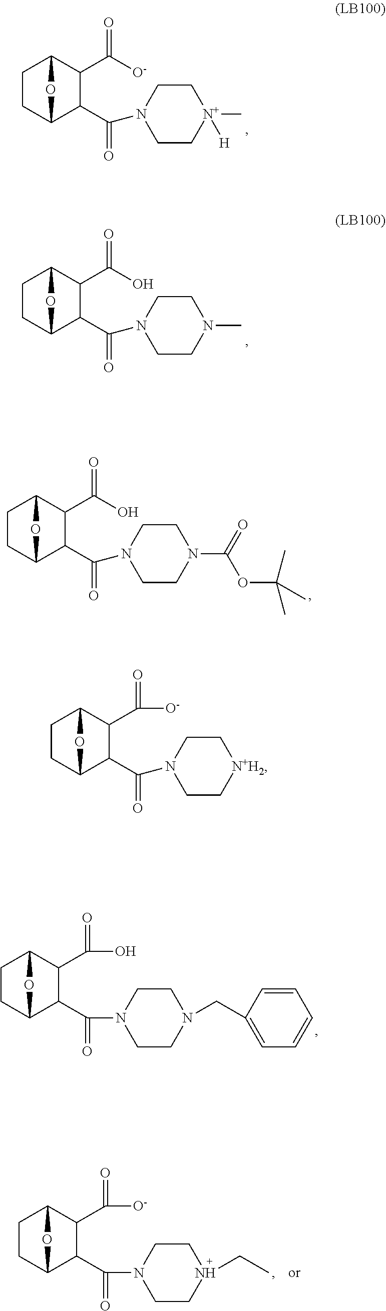

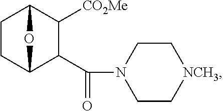 [SMILES: COC(=O)C1C(C(=O)N2CCN(C)CC2)[C@@H]2CC[C@H]1O2]

or a salt, zwitterion, or ester thereof.

In one embodiment, the compound of the method has the structure:

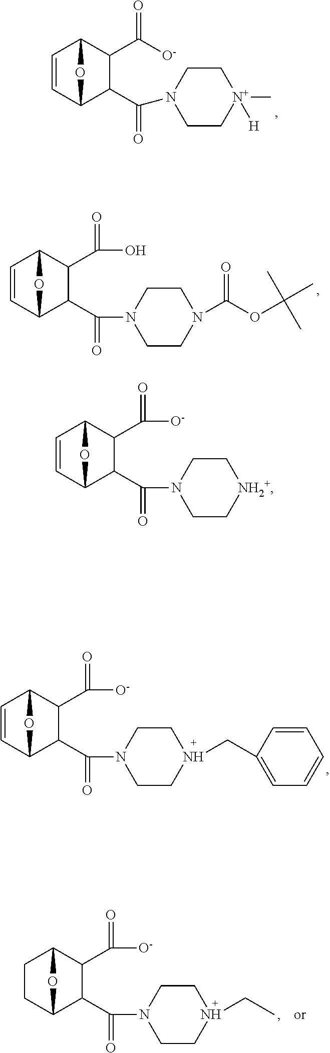

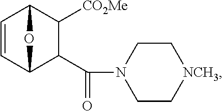 [SMILES: COC(=O)C1C(C(=O)N2CCN(C)CC2)[C@@H]2C=C[C@H]1O2]

or a salt, zwitterion, or ester thereof.

In one embodiment, the compound of the method has the structure:

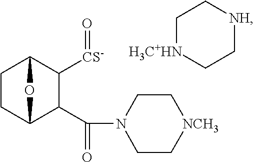

or a salt, zwitterion, or ester thereof.

In some embodiments of any of the above methods, the compound has the structure:

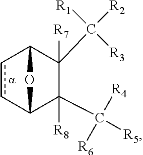

wherein

bond α is present or absent;

R₁ and R₂ together are ═O;

R₃ and R₄ are each different, and each is O(CH₂)₁₋₆R₉ or OR₉,

or

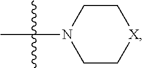

where X is O, S, NR₁₀, N⁺HR₁₀ or N⁺R₁₀R₁₀,
where each R₉ is H, alkyl, C₂-C₁₂ alkyl substituted alkyl, alkenyl, alkynyl, aryl, (C₆H₅)(CH₂)₁₋₆ (CHNHBOC)CO₂H, (C₆H₅)(CH₂)₁₋₆(CHNH₂)CO₂H, (CH₂)₁₋₆(CHNHBOC)CO₂H, (CH₂)₁₋₆(CHNH₂)CO₂H or (CH₂)₁₋₆CCl₃,
where each R₁₀ is independently H, alkyl, hydroxyalkyl, C₂-C₁₂ alkyl, alkenyl, C₄-C₁₂ alkenyl, alkynyl, aryl,

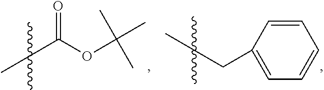

—CH₂CN, —CH₂CO₂R₁₁, or —CH₂COR₁₁,
where each R₁₁ is independently alkyl, alkenyl or alkynyl, each of which is substituted or unsubstituted, or H;
or R₃ and R₄ are each different and each is OH or

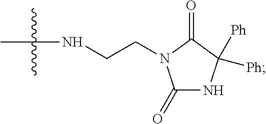 [SMILES: *NCCN1C(=O)NC(c2ccccc2)(c2ccccc2)C1=O]

R₅ and R₆ taken together are ═O;
R₇ and R₈ are each H; and
each occurrence of alkyl, alkenyl, or alkynyl is branched or unbranched, unsubstituted or substituted,
or a salt, zwitterion, or ester thereof.

In one embodiment, the compound of the method has the structure:

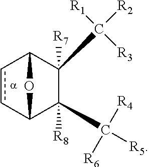

In one embodiment, the bond α is present.

In one embodiment, the bond α is absent.

In one embodiment, R₃ is OR₉ or O(CH₂)₁₋₆R₉, where R₉ is aryl, substituted ethyl or substituted phenyl, wherein the substituent is in the para position of the phenyl; R₄ is

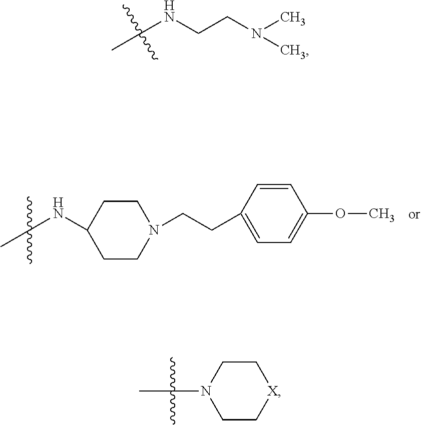

where X is O, S, NR₁₀, or N⁺R₁₀R₁₀,

where each R₁₀ is independently H, alkyl, hydroxyalkyl, substituted C₂-C₁₂ alkyl, alkenyl, substituted C₄-C₁₂ alkenyl, alkynyl, substituted alkynyl, aryl,

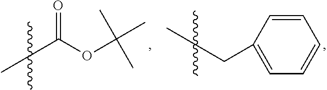

—CH₂CN, —CH₂CO₂R₁₁, —CH₂COR₁₁,

where R₁₁ is alkyl, alkenyl or alkynyl, each of which is substituted or unsubstituted, or H;
or where R₃ is OH and R₄ is

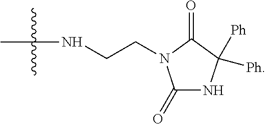 [SMILES: *NCCN1C(=O)NC(c2ccccc2)(c2ccccc2)C1=O]

In one embodiment, R₄ is

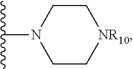

where R₁₀ is alkyl or hydroxylalkyl.

In one embodiment, R₁ and R₂ together are ═O; R₃ is OR₉ or O(CH₂)₁₋₂R₉, where R₉ is aryl, substituted ethyl, or substituted phenyl, wherein the substituent is in the para position of the phenyl; R₄ is

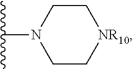

where R₁₀ is alkyl or hydroxyl alkyl; R₅ and R₆ together are ═O; and R₇ and R₈ are each independently H.

In one embodiment, R₁ and R₂ together are ═O; R₃ is O(CH₂)R₉, or OR₉, where R₉ is phenyl or CH₂CCl₃,

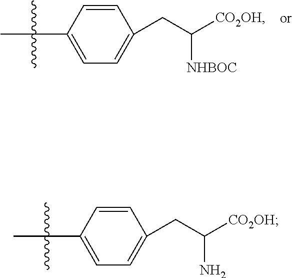

R₄ is

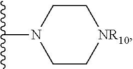

where R₁₀ is CH₃ or CH₃CH₂OH;

R₅ and R₆ together are ═O; and

R₇ and R₈ are each independently H.

In one embodiment, R₃ is OR₉, where R₉ is (CH₂)₁₋₆ (CHNHBOC)CO₂H, (CH₂)₁₋₆(CHNH₂)CO₂H, or (CH₂)₁₋₆CCl₃

In one embodiment, R₉ is CH₂(CHNHBOC)CO₂H, CH₂(CHNH₂)CO₂H, or CH₂CCl₃.

In one embodiment, R₉ is (C₆H₅)(CH₂)₁₋₆(CHNHBOC)CO₂H or (C₆H₅)(CH₂)₁₋₆(CHNH₂)CO₂H.

In one embodiment, R₉ is (C₆H₅)(CH₂)(CHNHBOC)CO₂H or (C₆H₅)(CH₂)(CHNH₂)CO₂H.

In one embodiment, R₃ is O(CH₂)₁₋₆R₉ or O(CH₂)R₉, where R₉ is phenyl.

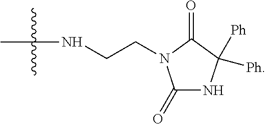 [SMILES: *NCCN1C(=O)NC(c2ccccc2)(c2ccccc2)C1=O]

In one embodiment, R₃ is OH and R₄ is

In one embodiment, R₄ is

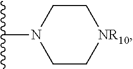

wherein R₁₀ is alkyl or hydroxyalkyl.

In one embodiment, R₁₁ is —CH₂CH₂OH or —CH₃.

In one embodiment, the compound has the structure:

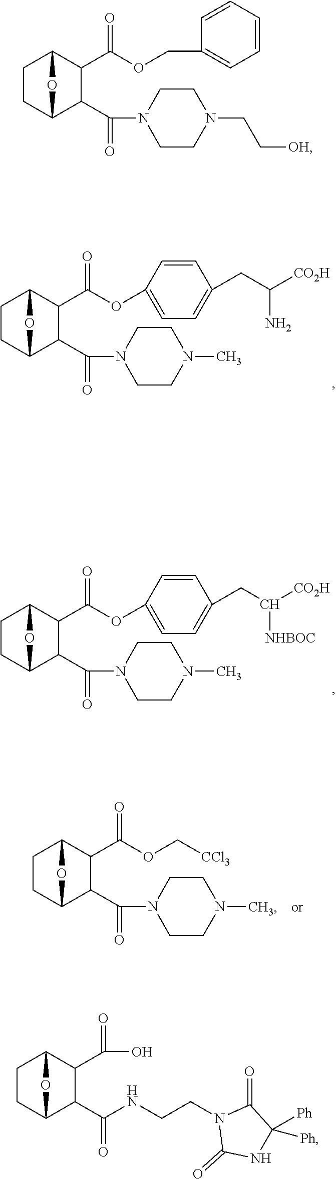

or a salt, zwitterion, or ester thereof.

In one embodiment, the compound has the structure:

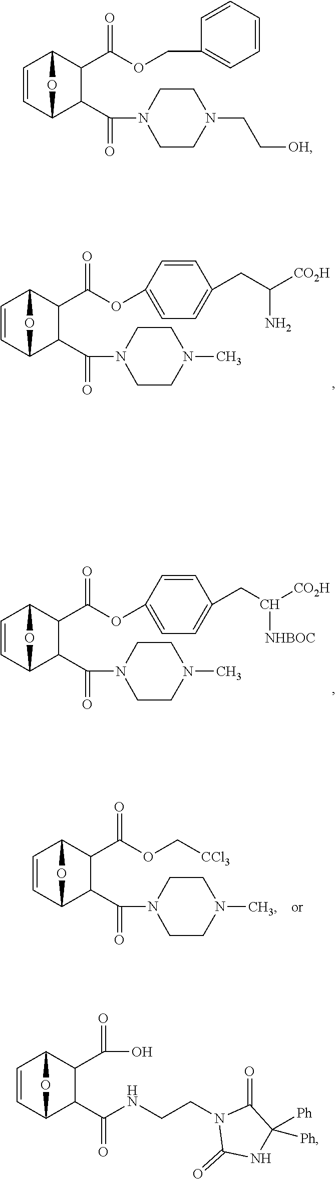

or a salt, zwitterion, or ester thereof.

In some embodiments of any of the above methods, the compound has the structure:

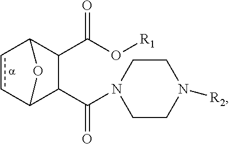

wherein
bond α is absent or present;
R₁ is C₂-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
or a salt, zwitterion, or ester thereof.

In some embodiments of any of the above methods, the compound has the structure:

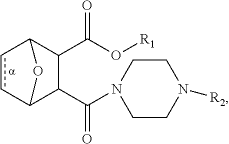

wherein
bond α is absent or present;
R₁ is C₃-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃, or a salt, zwitterion, or ester thereof.

In some embodiments of any of the above methods, the compound has the structure:

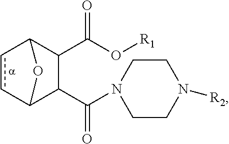

wherein
bond α is absent or present;
R₁ is C₄-C₂₀ alkyl, C₂-C₂₀ alkenyl, or C₂-C₂₀ alkynyl;

R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,

or a salt, zwitterion, or ester thereof.

In some embodiments of any of the above methods, the above compound having the structure:

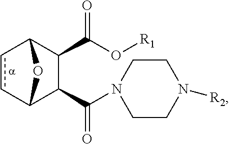

or a salt, zwitterion, or ester thereof.

In some embodiments, R₁ is

—CH₂CH₃,

—CH₂CH₂CH₃,

—CH₂CH₂CH₂CH₃,

—CH₂CH₂CH₂CH₂CH₃,

—CH₂CH₂CH₂CH₂CH₂CH₃,

—CH₂CH₂CH₂CH₂CH₂CH₂CH₃,

—CH₂CH₂CH₂CH₂CH₂CH₂CH₂CH₃,

—CH₂CH₂CH₂CH₂CH₂CH₂CH₂CH₂CH₃, or

—CH₂CH₂CH₂CH₂CH₂CH₂CH₂CH₂CH₂CH₃.

In some embodiments, R₁ is —CH₂CH₂CH₂CH₂CH₂CH₂CH₂CH₂CH₂CH₂CH₂CH₂CH₂CH₂CH₂CH₂CH₃, or —CH₂CH₂CH₂CH₂CH₂CH₂CH₂CH₂CH═CHCH₂CH═CHCH₂CH₂CH₂CH₂CH₃.

In some embodiments, R₂ is —H, —CH₃, —CH₂CH₃, —CH₂-phenyl, —CH₂CH₂—OH, or —C(O)C(CH₃)₃.

In some embodiments, the compound has the structure:

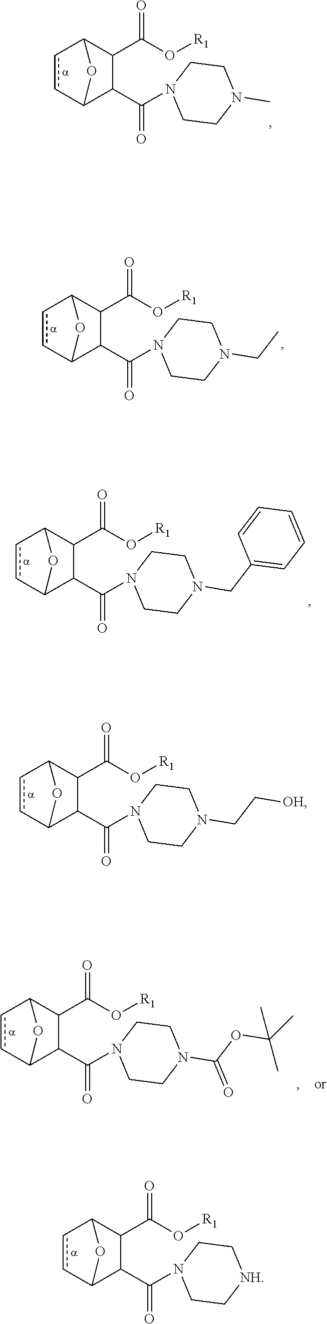

In some embodiments, the bond α is absent.

In some embodiments, the bond α is present.

In some embodiments, the compound has the structure:

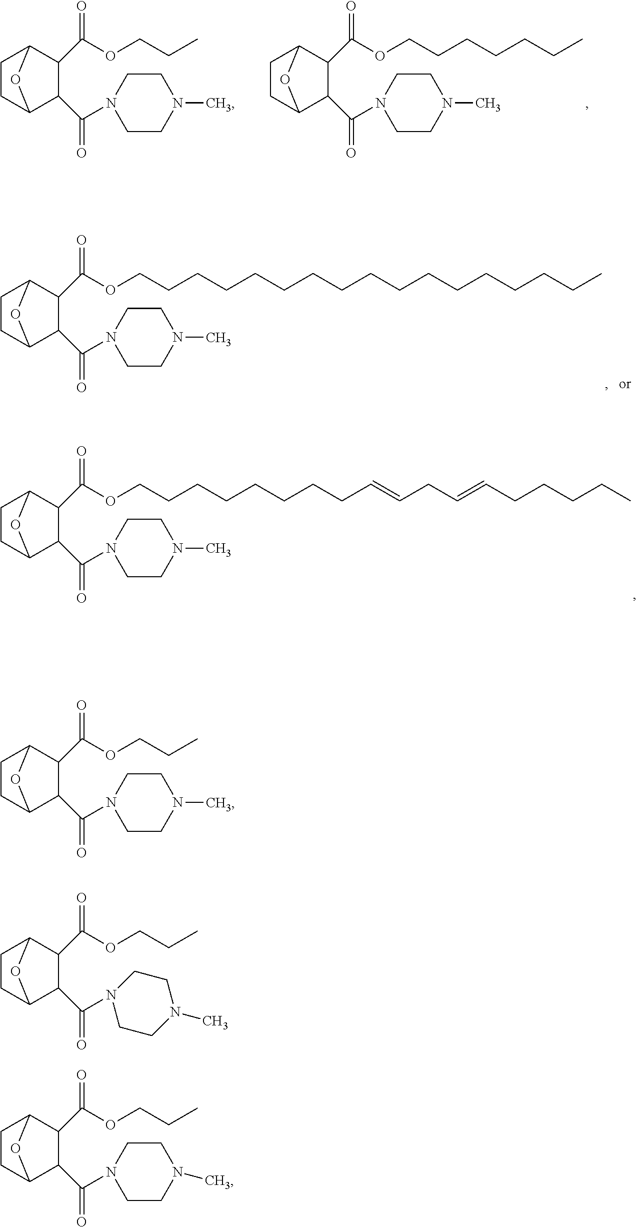

or a salt, zwitterion, or ester thereof.

The analogs of LB-100 disclosed herein have analogous activity to LB-100 and work similarly in the methods described herein.

In some embodiments of any of the above methods, the subject is administered a pharmaceutical composition comprising a compound of the present invention and at least one pharmaceutically acceptable carrier for treating the sAML in the subject.

In some embodiments, the pharmaceutically acceptable carrier comprises a liposome.

In some embodiments, the compound is contained in a liposome or microsphere.

In some embodiments, the pharmaceutical composition comprises the PP2A inhibitor and the anti-cancer agent.

In some embodiments of any of the above methods or uses, the subject is a human.

In some embodiments of any of the above methods or uses, the compound and/or the anti-cancer agent is orally administered to the subject.

The present invention provides a PP2A inhibitor for use in treating sAML.

The present invention provides a PP2A inhibitor for use in treating sAML in a subject afflicted with sAML.

The present invention provides a PP2A inhibitor in combination with an anti-cancer agent for use in treating a subject afflicted with sAML.

The present invention provides a PP2A inhibitor for use in enhancing cytotoxicity of an anti-cancer agent in treating sAML.

The present invention provides a PP2A inhibitor for use in enhancing cytotoxicity of daunorubicin in a subject afflicted with sAML.

The present invention provides a PP2A inhibitor for use in enhancing cytotoxicity of daunorubicin in a subject afflicted with sAML via upregulation of miR-181b-1.

The present invention provides use of a PP2A inhibitor for treating sAML.

The present invention provides use of a PP2A inhibitor for treating sAML in a subject afflicted with sAML.

The present invention provides use of a PP2A inhibitor for enhancing cytotoxic activity of an anti-cancer agent.

The present invention provides use of a PP2A inhibitor for enhancing cytotoxic activity of daunorubicin.

The present invention provides use of a PP2A inhibitor for enhancing cytotoxic activity of daunorubicin via upregulation of miR-181b-1.

In some embodiments, the PP2A inhibitor is LB100.

In some embodiments, the invention provides a method of treating sAML in a subject comprising administering to said subject (a) a PP2A inhibitor, such as LB100 or a pharmaceutically acceptable salt thereof, in an amount which is therapeutically effective at treating sAML.

In some embodiments, the invention provides the use of (a) a PP2A inhibitor, such as LB100 or a pharmaceutically acceptable salt thereof, for the preparation of a medicament for the treatment of sAML.

In some embodiments, the invention provides the use of (a) a PP2A inhibitor, such as LB100 or a pharmaceutically acceptable salt thereof, for treating sAML.

In some embodiments, the present invention presents a method of treating sAML in a patient comprising administering to the patient (a) LB100, or a pharmaceutically acceptable salt thereof; and (b) daunorubicin, or a pharmaceutically acceptable salt thereof.

In some embodiments, the initial dose of LB100 administered to the subject is an amount of from 0.1 mg/m² to 5 mg/m².

In some embodiments, the further dose of LB100 administered to the subject is an amount of from 0.1 mg/m² to 5 mg/m².

In some embodiments, the compound is administered at a dose of 0.25 mg/m², 0.5 mg/m², 0.83 mg/m², 1.25 mg/m², 1.75 mg/m², 2.33 mg/m², of 3.1 mg/m².

In some embodiments, the compound is administered at a dose of 2.33 mg/m².

In some embodiments, the compound is administered for 3 days every 3 weeks.

In some embodiments, the further dose of LB100 administered to the subject is an amount 25% less than the initial dose.

In some embodiments, the further dose of LB100 administered to the subject is an amount 50% less than the initial dose.

In some embodiments, the further dose of LB100 administered to the subject is an amount 75% less than the initial dose.

In some embodiments, the further dose of LB100 administered to the subject is an amount 25% more than the initial dose.

In some embodiments, the further dose of LB100 administered to the subject is an amount 50% more than the initial dose.

In some embodiments, the further dose of LB100 administered to the subject is an amount 75% more than the initial dose.

In some embodiments, the anti-cancer agent is administered in a dosage range of about 1.0-1000.0 mg/m². In some embodiments, the anti-cancer agent is administered in a dosage range of about 100.0-750.0 mg/m², about 200.0-600.0 mg/m², or about 200.0-500.0 mg/m². In some embodiments, the anti-cancer agent is administered at a dosage of about 200.0 mg/m², about 250.0 mg/m², about 300.0 mg/m², about 350.0 mg/m², about 400.0 mg/m², about 450.0 mg/m², or about 500.0 mg/m². In some embodiments, the anti-cancer agent is administered at a dosage of about 500.0 mg/m².

In some embodiments, the subject is further treated with an anti-cancer therapy concurrently with, prior to, or after the administration of the PP2A inhibitor.

In some embodiments, daunorubicin, or a pharmaceutically acceptable salt thereof, is administered in a dosage range of about 1.0-1000.0 mg/m². In some embodiments, daunorubicin, or a pharmaceutically acceptable salt thereof, is administered in a dosage range of about 100.0-750.0 mg/m², about 200.0-600.0 mg/m², or about 200.0-500.0 mg/m². In some embodiments, daunorubicin, or a pharmaceutically acceptable salt thereof, is administered at a dosage of about 200.0 mg/m², about 250.0 mg/m², about 300.0 mg/m², about 350.0 mg/m², about 400.0 mg/m², about 450.0 mg/m², or about 500.0 mg/m². In some embodiments, daunorubicin, or a pharmaceutically acceptable salt thereof, is administered at a dosage of about 500.0 mg/m².

In some embodiments, the subject is further treated with daunorubicin, or a pharmaceutically acceptable salt thereof, concurrently with, prior to, or after the administration of the PP2A inhibitor.

In some embodiments, the PP2A inhibitor, such as LB 100 or a pharmaceutically acceptable salt thereof, is administered via an intravenous infusion. In some embodiments, an intravenous infusion of the PP2A inhibitor is about 30 minutes to about 5 hours, about 30 minutes to about 4 hours, about 30 minutes to about 3 hours, about 30 minutes to about 2 hours, or about 30 minutes to about 1.5 hours. In some embodiments, an intravenous infusion of the PP2A inhibitor is about 30, 40, 50, or 60 minutes. In some embodiments, an intravenous infusion of the PP2A inhibitor is about 1.5, 2, 2.5, 3, 3.5, 4, 4.5, or 5 hours. In some embodiments, an intravenous infusion of the PP2A inhibitor is about one hour.

In some embodiments, the anti-cancer agent, such as daunorubicin or a pharmaceutically acceptable salt thereof, is administered via intravenous infusion. In some embodiments, an intravenous infusion of the anti-cancer agent is about 1 minute to about 1 hour. In some embodiments, an intravenous infusion of the anti-cancer agent is about 1-40 minutes, about 1-30 minutes, about 1-20 minutes, or about 5-15 minutes. In some embodiments, an intravenous infusion of the anti-cancer agent is about 5, 6, 7, 8, 9, 10, 11, 12, 13, 14, or 15 minutes. In some embodiments, an intravenous infusion of the anti-cancer agent is about 10 minutes.

In some embodiments, the invention provides a method of treating sAML in a subject comprising administering to said subject (a) LB100, or a pharmaceutically acceptable salt thereof; and (b) daunorubicin, or a pharmaceutically acceptable salt thereof, in an amount which is therapeutically effective or jointly therapeutically effective at treating sAML.

In some embodiments, the invention provides the use of (a) LB100, or a pharmaceutically acceptable salt thereof; and (b) daunorubicin, or a pharmaceutically acceptable salt thereof, for the preparation of a medicament for the treatment of sAML.

In some embodiments, the invention provides the use of (a) LB100, or a pharmaceutically acceptable salt thereof; and (b) daunorubicin, or a pharmaceutically acceptable salt thereof, for treating sAML.

In some embodiments, a benefit of the use of the combination of (a) LB100, or a pharmaceutically acceptable salt thereof; and (b) daunorubicin, or a pharmaceutically acceptable salt thereof, when used in combination can show synergy when compared to LB100 as a monotherapy or to daunorubicin as a monotherapy.

It can be shown by established test models that administration of (a) LB100, or a pharmaceutically acceptable salt thereof; and (b) daunorubicin, or a pharmaceutically acceptable salt thereof, results in the beneficial effects described herein. The person skilled in the art is fully enabled to select a relevant test model to prove such beneficial effects. The pharmacological activity of a compound or combination may, for example, be demonstrated in a clinical study or in an in vivo or in vitro test procedure as known in the art or as described hereinafter.

In one aspect, the invention provides a pharmaceutical composition comprising a quantity, which is jointly therapeutically effective at treating sAML in a subject, of (a) LB100, or a pharmaceutically acceptable salt thereof; and (b) daunorubicin, or a pharmaceutically acceptable salt thereof. In this composition, the combination partners (a) and (b) are administered in a single formulation or unit dosage form by any suitable route. The unit dosage form may also be a fixed combination.

In a further aspect, the invention provides pharmaceutical compositions separately comprising a quantity, which is jointly therapeutically effective at treating mesothelioma in a subject, of (a) LB100, or a pharmaceutically acceptable salt thereof; and (b) daunorubicin, or a pharmaceutically acceptable salt thereof, which are administered concurrently but separately, or administered sequentially.

The pharmaceutical compositions for separate administration of the combination partners, or for the administration in a fixed combination, e.g., a single galenical composition comprising (a) LB100, or a pharmaceutically acceptable salt thereof; and (b) daunorubicin, or a pharmaceutically acceptable salt thereof, may be prepared in a manner known in the art and are those suitable for enteral (such as oral or rectal) and/or parenteral administration to subjects and comprising a therapeutically effective amount of at least one combination partner alone, e.g. as indicated above, or in combination with one or more pharmaceutically acceptable carriers.

The novel pharmaceutical composition may contain from about 0.1% to about 99.9%, for example from about 1% to about 60%, of the active ingredient(s).

Pharmaceutical compositions comprising a disclosed compound or combination, including fixed combinations or non-fixed combinations, for enteral or parenteral administration are, for example, those in unit dosage forms, such as sugar-coated tablets, tablets, capsules or suppositories, or ampoules. If not indicated otherwise, these are prepared in a manner known in the art, for example by means of various conventional mixing, comminution, granulating, sugar-coating, dissolving, lyophilizing processes, or fabrication techniques readily apparent to those skilled in the art. It will be appreciated that the unit content of a combination partner contained in an individual dose of each dosage form need not in itself constitute an effective amount since the necessary effective amount may be reached by administration of a plurality of dosage units. It will be further appreciated that the unit content of a combination partner for parenteral administration may contain a higher dosage amount of the combination partner which is diluted to the effective dosage amount before administration.

A unit dosage form containing the combination of agents or individual agents of the combination of agents may be in the form of micro-tablets enclosed inside a capsule, e.g. a gelatin capsule. For this, a gelatin capsule as is employed in pharmaceutical formulations can be used, such as the hard gelatin capsule known as CAPSUGEL™, available from Pfizer.

The unit dosage forms of the present invention may optionally further comprise additional conventional carriers or excipients used for pharmaceuticals. Examples of such carriers include, but are not limited to, disintegrants, binders, lubricants, glidants, stabilizers, and fillers, diluents, colorants, flavors, and preservatives. One of ordinary skill in the art may select one or more of the aforementioned carriers with respect to the particular desired properties of the dosage form by routine experimentation and without any undue burden. The amount of each carrier used may vary within ranges conventional in the art. The following references which are all hereby incorporated by reference disclose techniques and excipients used to formulate oral dosage forms. See The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2003).

These optional additional conventional carriers may be incorporated into the oral dosage form either by incorporating the one or more conventional carriers into the initial mixture before or during melt granulation or by combining the one or more conventional carriers with the granules in the oral dosage form. In the latter embodiment, the combined mixture may be further blended, e.g., through a V-blender, and subsequently compressed or molded into a tablet, for example a monolithic tablet, encapsulated by a capsule, or filled into a sachet.

Examples of pharmaceutically acceptable disintegrants include, but are not limited to, starches; clays; celluloses; alginates; gums; cross-linked polymers, e.g., cross-linked polyvinyl pyrrolidone or crospovidone, e.g., POLYPLASDONE XL™ from International Specialty Products (Wayne, N.J.); cross-linked sodium carboxymethylcellulose or croscarmellose sodium, e.g., AC-DI-SOL™ from FMC; and cross-linked calcium carboxymethylcellulose; soy polysaccharides; and guar gum. The disintegrant may be present in an amount from about 0% to about 10% by weight of the composition. In one embodiment, the disintegrant is present in an amount from about 0.1% to about 5% by weight of composition.

Examples of pharmaceutically acceptable binders include, but are not limited to, starches; celluloses and derivatives thereof, for example, microcrystalline cellulose, e.g., AVICEL PH™ from FMC (Philadelphia, Pa.), hydroxypropyl cellulose hydroxylethyl cellulose and hydroxylpropylmethyl cellulose METHOCEL™ from Dow Chemical Corp. (Midland, Mich.); sucrose; dextrose; corn syrup; polysaccharides; and gelatin. The binder may be present in an amount from about 0% to about 50%, e.g., 2-20% by weight of the composition.

Examples of pharmaceutically acceptable lubricants and pharmaceutically acceptable glidants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. The lubricant may be present in an amount from about 0% to about 10% by weight of the composition. In one embodiment, the lubricant may be present in an amount from about 0.1% to about 1.5% by weight of composition. The glidant may be present in an amount from about 0.1% to about 10% by weight.

Examples of pharmaceutically acceptable fillers and pharmaceutically acceptable diluents include, but are not limited to, confectioner's sugar, compressible sugar, dextrates, dextrin, dextrose, lactose, mannitol, microcrystalline cellulose, powdered cellulose, sorbitol, sucrose and talc. The filler and/or diluent, e.g., may be present in an amount from about 0% to about 80% by weight of the composition.

The optimum ratios, individual and combined dosages, and concentrations of the therapeutic agent or agents that yield efficacy without toxicity are based on the kinetics of the therapeutic agent's availability to target sites, and are determined using methods known to those of skill in the art.

In accordance with the present invention, a therapeutically effective amount of each of (a) LB100, or a pharmaceutically acceptable salt thereof; and (b) daunorubicin, or a pharmaceutically acceptable salt thereof, may be administered simultaneously or sequentially and in any order, and the components may be administered separately or as a fixed combination. For example, in one aspect the invention provides a method of preventing or treating sAML may comprise (i) administration of the first agent (a) in free or pharmaceutically acceptable salt form, and (ii) administration of an agent (b) in free or pharmaceutically acceptable salt form, simultaneously or sequentially in any order, in jointly therapeutically effective amounts, in some embodiments in synergistically effective amounts, e.g., in daily or intermittent dosages corresponding to the amounts described herein. The individual therapeutic agents may be administered separately at different times during the course of therapy or concurrently in divided or single combination forms. Furthermore, the term “administering” also encompasses the use of a pro-drug of a therapeutic agent that converts in vivo to the therapeutic agent. The instant invention is therefore to be understood as embracing all such regimens of simultaneous or alternating treatment and the term “administering” is to be interpreted accordingly.

The effective dosage of each of the therapeutic agents or combination thereof may vary depending on the particular therapeutic agent or pharmaceutical composition employed, the mode of administration, the condition being treated, and the severity of the condition being treated. Thus, the dosage regimen is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition.

In embodiments where at least two therapeutic agents are used in combination, the effective dosage of each of the therapeutic agents may require more frequent administration of one of the therapeutic agent(s) as compared to the other therapeutic agent(s) in the combination. Therefore, to permit appropriate dosing, packaged pharmaceutical products may contain one or more dosage forms that contain the combination of compounds, and one or more dosage forms that contain one of the combination of therapeutic agent(s), but not the other therapeutic agent(s) of the combination.

When the combination of therapeutic agents, such as a combination of (a) LB 100, or a pharmaceutically acceptable salt thereof; and (b) daunorubicin, or a pharmaceutically acceptable salt thereof, are applied in the form as marketed as single drugs, their dosage and mode of administration can be in accordance with the information provided on the package insert of the respective marketed drug, if not mentioned herein otherwise.

In some embodiments, (a) LB 100, or a pharmaceutically acceptable salt thereof; and (b) daunorubicin, or a pharmaceutically acceptable salt thereof, is administered twice per day, once per day, once every two days, once every three days, once every four days, once every five days, once every six days, once a week, once every two weeks, once every three weeks, once every four weeks, once a month, once every two months, once every three months, once every four months, once every six months, or once per year.

The optimal dosage of each therapeutic agent for promotion and/or enhancement of an immune response in a subject and/or treating sAML in a subject can be determined empirically for each individual using known methods and will depend upon a variety of factors, including, though not limited to, the degree of advancement of the disease; the age, body weight, general health, gender and diet of the individual; the time and route of administration; and other medications the individual is taking. Optimal dosages may be established using routine testing and procedures that are well known in the art.

The amount of each therapeutic agent that may be combined with the carrier materials to produce a single dosage form will vary depending upon the individual treated and the particular mode of administration. In some embodiments the unit dosage forms containing the combination of therapeutic agents as described herein will contain the amounts of each agent of the combination that are typically administered when the therapeutic agents are administered alone.

Frequency of dosage may vary depending on the therapeutic agent used and the particular condition to be treated. In general, the use of the minimum dosage that is sufficient to provide effective therapy is preferred. Patients may generally be monitored for therapeutic effectiveness using assays suitable for the condition being treated, which will be familiar to those of ordinary skill in the art.

In some embodiments, the PP2A inhibitor has the structure

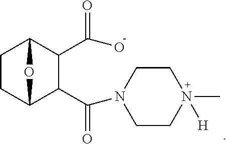

In some embodiments, the method further comprises administering one or more additional anti-cancer agents, such as daunorubicin.

The present invention also provides a method of treating a subject afflicted with sAML comprising administering to the subject an effective amount of a PP2A inhibitor in combination with an effective amount of an anti-cancer therapy, wherein the amounts when taken together are effective to treat the subject.

The present invention also provides a method of treating a subject afflicted with sAML and receiving anti-cancer therapy comprising administering to the subject an effective amount of PP2A inhibitor effective to enhance treatment relative to the anti-cancer therapy alone.

The compounds used in the method of the present invention are protein phosphatase 2A (PP2A) inhibitors. Methods of preparation may be found in Lu et al., 2009; U.S. Pat. No. 7,998,957 B2; and U.S. Pat. No. 8,426,444 B2. Compound LB-100 is an inhibitor of PP2A in vitro in human cancer cells and in xenografts of human tumor cells in mice when given parenterally in mice. LB-100 inhibits the growth of cancer cells in mouse model systems.

### 2. Definitions

The general terms used herein are defined with the following meanings, unless explicitly stated otherwise:

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “combination” or “pharmaceutical combination” is defined herein to refer to either a fixed combination in one dosage unit form, a non-fixed combination or a kit of parts for the combined administration where the PP2A inhibitor, such as LB100 or a pharmaceutically acceptable salt thereof, and an additional anti-cancer agent, such as daunorubicin or a pharmaceutically acceptable salt thereof, may be administered independently at the same time or separately within time intervals that allow that the combination partners show a cooperative, e.g., synergistic, effect.

The term “fixed combination” means that the active ingredients or therapeutic agents, e.g., LB100, or a pharmaceutically acceptable salt thereof, and an anti-cancer agent, are administered to a patient simultaneously in the form of a single entity or dosage form.

The term “non-fixed combination” means that the active ingredients or therapeutic agents, e.g., LB100, or a pharmaceutically acceptable salt thereof, and an anti-cancer agent, are administered to a patient as separate entities or dosage forms either simultaneously, concurrently or sequentially with no specific time limits, wherein such administration provides therapeutically effective levels of the compounds in the body of the subject, e.g., a mammal or human, in need thereof.

The term “pharmaceutical composition” is defined herein to refer to a mixture or solution containing at least one therapeutic agent to be administered to a subject, e.g., a mammal or human, in order to treat a particular disease or condition affecting the subject thereof.

The term “pharmaceutically acceptable” is defined herein to refer to those compounds, biologic agents, materials, compositions and/or dosage forms, which are, within the scope of sound medical judgment, suitable for contact with the tissues a subject, e.g., a mammal or human, without excessive toxicity, irritation allergic response and other problem complications commensurate with a reasonable benefit/risk ratio.

The terms “combined administration” as used herein are defined to encompass the administration of the selected therapeutic agents to a single subject, e.g., a mammal or human, and are intended to include treatment regimens in which the agents are not necessarily administered by the same route of administration or at the same time.

The term “treating” or “treatment” as used herein comprises a treatment relieving, reducing or alleviating at least one symptom in a subject or affecting a delay of progression of a disease, condition and/or disorder. For example, treatment can be the diminishment of one or several symptoms of a disorder or complete eradication of a disorder. Within the meaning of the present invention, the term “treat” also denotes to arrest, delay the onset (e.g., the period prior to clinical manifestation of a disease) and/or reduce the risk of developing or worsening a disease.

The term “jointly therapeutically active” or “joint therapeutic effect” as used herein means that the therapeutic agents may be given separately (in a chronologically staggered manner, for example in a sequence-specific manner) such that the warm-blooded animal (for example, human) to be treated, still shows an interaction, such as a synergistic interaction (joint therapeutic effect). Whether this is the case can, inter alia, be determined by following the blood levels, showing that both therapeutic agents are present in the blood of the human to be treated at least during certain time intervals.

An “effective amount”, “pharmaceutically effective amount”, or “therapeutically effective amount” of a therapeutic agent is an amount sufficient to provide an observable improvement over the baseline clinically observable signs.

The term “synergistic effect” as used herein refers to action of two or more agents, for example, (a) LB100, or a pharmaceutically acceptable salt thereof, and (b) an anti-cancer agent, producing an effect, for example, promoting and/or enhancing treatment of cancer in a subject, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The term “subject” or “patient” as used herein includes animals, such as mammals, e.g., humans, dogs, cows, horses, pigs, sheep, goats, cats, mice, rabbits, rats and transgenic non-human animals. In some embodiments, the subject is a human, e.g., a human suffering from mesothelioma or pleural malignant mesothelioma.

The term “about” or “approximately” shall have the meaning of within 10%, for example within 5%, of a given value or range.

The structure of the active ingredients identified by code numbers, generic or trade names may be taken from the actual edition of the standard compendium “The Merck Index” or from databases, e.g., Patents International (e.g., IMS World Publications). The corresponding content thereof is hereby incorporated by reference.

As used herein, a “symptom” associated with reperfusion injury includes any clinical or laboratory manifestation associated with reperfusion injury and is not limited to what the subject can feel or observe.

As used herein, “treatment of the diseases” or “treating”, e.g. of reperfusion injury, encompasses inducing prevention, inhibition, regression, or stasis of the disease or a symptom or condition associated with the disease.

As used herein, “inhibition” of disease progression or disease complication in a subject means preventing or reducing the disease progression and/or disease complication in the subject.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₂₀ alkyl, C₂-C₂₀ alkyl, C₃-C₂₀ alkyl, C₄-C₂₀ alkyl and so on. An embodiment can be C₁-C₃₀ alkyl, C₂-C₃₀ alkyl, C₃-C₃₀ alkyl, C₄-C₃₀ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl, C₃-C₁₂ alkenyl, C₂-C₂₀ alkenyl, C₃-C₂₀ alkenyl, C₂-C₃₀ alkenyl, or C₃-C₃₀ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₁₂ alkynyl, C₂-C₂₀ alkynyl, C₃-C₂₀ alkynyl, C₂-C₃₀ alkynyl, or C₃-C₃₀ alkynyl.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynl, or aryl groups as hereinabove defined.

Each occurrence of alkyl, alkenyl, or alkynyl is branched or unbranched, unsubstituted or substituted.

The alkyl, alkenyl, alkynyl, and aryl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, and alkynyl groups can be further substituted by replacing one or more hydrogen atoms by non-hydrogen groups described herein to the extent possible. These include, but are not limited to, halo, hydroxy, mercapto, amino, carboxy, cyano and carbamoyl.

The term “substituted” as used herein means that a given structure has a substituent which can be an alkyl, alkenyl, or aryl group as defined above. The term shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

Other injectable drug delivery systems include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The compounds used in the method of the present invention may be in a salt form. As used herein, a “salt” is a salt of the instant compounds which has been modified by making acid or base salts of the compounds. In the case of compounds used to treat an infection or disease, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The present invention includes esters or pharmaceutically acceptable esters of the compounds of the present method. The term “ester” includes, but is not limited to, a compound containing the R—CO—OR′ group. The “R—CO—O” portion may be derived from the parent compound of the present invention. The “R′” portion includes, but is not limited to, alkyl, alkenyl, alkynyl, heteroalkyl, aryl, and carboxy alkyl groups.

The present invention includes pharmaceutically acceptable prodrug esters of the compounds of the present method. Pharmaceutically acceptable prodrug esters of the compounds of the present invention are ester derivatives which are convertible by solvolysis or under physiological conditions to the free carboxylic acids of the parent compound. An example of a pro-drug is an alkly ester which is cleaved in vivo to yield the compound of interest.

The compound, or salt, zwitterion, or ester thereof, is optionally provided in a pharmaceutically acceptable composition including the appropriate pharmaceutically acceptable carriers,

As used herein, an “amount” or “dose” of an agent measured in milligrams refers to the milligrams of agent present in a drug product, regardless of the form of the drug product.

The National Institutes of Health (NIH) provides a table of Equivalent Surface Area Dosage Conversion Factors below (Table A) which provides conversion factors that account for surface area to weight ratios between species.

TABLE A
Equivalent Surface Area Dosage Conversion Factors

| | To | | | | | |
|---|---|---|---|---|---|---|
| | | Mouse | Rat | Monkey | Dog | Man |
| From | | 20 g | 150 g | 3 kg | 8 kg | 60 kg |
| | Mouse | 1 | ½ | ¼ | ⅙ | 1/12 |
| | Rat | 2 | 1 | ½ | ¼ | 1/7 |
| | Monkey | 4 | 2 | 1 | ⅗ | ⅓ |
| | Dog | 6 | 4 | 1 ⅔ | 1 | ½ |
| | Man | 12 | 7 | 3 | 2 | 1 |

As used herein, the term “therapeutically effective amount” or “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Where a range is given in the specification it is understood that the range includes all integers and 0.1 units within that range, and any sub-range thereof. For example, a range of 77 to 90% is a disclosure of 77, 78, 79, 80, and 81% etc.

As used herein, “about” with regard to a stated number encompasses a range of +one percent to −one percent of the stated value. By way of example, about 100 mg/kg therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/kg. Accordingly, about 100 mg/kg includes, in an embodiment, 100 mg/kg.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

For the foregoing embodiments, each embodiment disclosed herein is contemplated as being applicable to each of the other disclosed embodiments. Thus, all combinations of the various elements described herein are within the scope of the invention.

All features of each of the aspects of the invention apply to all other aspects mutatis mutandis.

This invention will be better understood by reference to the Experimental Details which follow, but those skilled in the art will readily appreciate that the specific experiments detailed are only illustrative of the invention as described more fully in the claims which follow thereafter.

### EXEMPLIFICATION

### General Procedures—Materials and Methods

Reagents.

A stock solution of LB100 (10 mM) was prepared in phosphate-buffered saline (PBS, KeYi, Hangzhou, China) and kept at −80° C. Daunorubicin (DNR) was purchased from Haizheng Pharmacia (Zhejiang, China) and stored at −80° C. miR-181b-1 inhibitor was purchased from JiMa (Shanghai, China).

Established Cell Lines and Primary Cell Culture.

The following five human leukemia cell lines were obtained from the Shanghai Institute of Cell Biology (Shanghai, China): Kasumi-1, HL-60, THP-1, U937 and K562. The SICM-1 leukemia cell line was acquired from the Health Science Research Resources Bank (Osaka, Japan), established from a patient with MDS that had progressed to myelomonocytic leukemia.

Bone marrow (BM) samples were obtained from three sAML patients prior to initiation of chemotherapy after obtaining their informed written consent. The samples were enriched for mononuclear cells (MNC) and cultured at 37° C. in RPMI 1640 medium supplemented with 10% heat-inactivated fetal bovine serum (Gibco, MT, USA) in a humidified atmosphere of 5% CO₂. The collection and analysis of patient samples were approved by the Ethical Committee of the First Affiliated Hospital of Zhejiang University, and written informed consent was obtained from all patients. All methods were carried out in accordance with the approved guidelines and regulations by the First Affiliated Hospital of Zhejiang University. All experimental protocols were approved by the First Affiliated Hospital of Zhejiang University.

PP2A Phosphatase Activity Assay.

1×IV SKM-1 cells were seeded into 6-well microtiter plates and treated with different concentrations of LB100 (0, 1.25, 2.5, 5, 10 μNI). Following treatment for 6 hours, cells were washed twice with cold water, and lysed in RIPA buffer supplemented with Complete Protease Inhibitor Cocktail (Roche, Mannhein, Germany) for 20 minutes on ice. Cell lysate was sonicated for 10 seconds and then centrifuged at 20,000 g for 15 minutes. Supernatant was then assayed with the PP2A Immunoprecipitation Phosphatase Assay Kit (Millipore, Mass., USA).

MTT Assay.

Cells were seeded into 96-well microtiter plates (Nunc, Roskilde, Denmark) at densities of either 1×10⁵ cells/ml (established cell lines) or 5×10⁵ cells/ml (primary AML cells). Cultures were exposed to different drugs for 24 h. After exposure, 20 μl of 3-(4, 5-dimethylthiazol-2-yl)-2, 5-diphenylterazolium bromide solution (MTT, Sigma-Aldrich) was added to each well. The plates were then incubated for 4 h at 37° C. The MTT-containing solution was then aspirated away, 200 μI DMSO added to each well, and absorbance at 570 nm was measured.

Assessment of Apoptosis.

Cells were seeded into 6-well plates, and treated for 24h at 37° C. with different concentrations of LB100 (0, 1.25, 2.5, 5,10 μM). After washing with PBS, aliquots of the cells were resuspended in binding buffer, and stained with 5 μl Annexin V and 5 μl propidium iodide (Biouniquer, Nanjing, China) according to the manufacturer's instructions. Fluorescence-activated cell sorting (FACS) was then performed immediately after staining.

Cells (5×10⁵ cells/well) were pre-incubated for 3 h at 37° C. in the presence or absence of 20 KM of the pan-caspase inhibitor z-VAD-fmk (R&D Systems, MN, USA) in DMSO. Cells were then treated with LB100 for 24h, and processed in the Annexin V-binding assay as described above.

Hoechst Staining.

SKM-1 cells were treated with LB100 for 24 h. Cells were then permeabilized with 0.5% Triton X-100 for 30 min, washed with PBS, stained with 10 μg/ml Hoechst for 30 min, and washed with PBS. Nuclear morphology was observed immediately after using a BX51 fluorescence microscope (Olympus, Tokyo, Japan).

Cell Cycle Analysis by Flow Cytometry.

Cells were collected after being fixed overnight in 75% ethanol at −20° C. Fixed cells were washed twice with PBS, then incubated for 30 min with RNase A and propidium iodide (10 μg/ml). Cell cycle analysis was performed using a BDL SRII Flow Cytometer and FACSDiva Software (BD Bioscience, Franklin Lakes, USA). Raw data was analyzed using ModFit LT 3.2 software (Verity Software House, Topsham, USA).

Leukemia Colony-Forming Assay.

LB100-treated SKM-1 cells were seeded in a methylcellulose medium and incubated for 7 d at 37° C. The number of leukemia colony-forming units (CFU-Ls) containing >50 cells were determined manually under a light microscope (Olympus).

Western Blotting.

Cells were washed twice in PBS and lysed in 10 mM Tris, 1 mM ethylenediaminetetra-acetic acid (EDTA), 10 mM KCl, 0.3% Triton, and 0.1 mM phenylmethanesulfonyl fluoride (PMSF). Equal amounts of protein (30-50 μg) were separated on 8-12% SDS-polyacrylamide gels, and transferred to polyvinylidene fluoride (PVDF) membranes. Membranes were blocked with 5% non-fat milk and incubated overnight with the appropriate primary antibody at manufacturer-specified dilutions. Primary monoclonal antibodies were against I3-ACTIN (Santa Cruz Biotechnology, CA, USA); CDCl₂₅C, p-CDCl₂₅C, CDCl₂, p-CDCl₂, PP2A-A, PP2A-B, PP2A-C (Epitomics, USA); AKT, p-AKT, PARP, RAS, p-MEK, p-ERK, Raf, p-CamKII, Bc.1-2 and p-Bd-2 (Cell Signaling Technology, MA, USA).

Next, the membranes were washed three times in TBS-T buffer (10 mm Tris-HCl, pH 8, 150 mm NaCl, 0.1% Tween 20), and incubated for 1 h with the corresponding horseradish peroxidase (HRP)-conjugated secondary antibody at 1:5,000 dilution. Bound secondary antibody was detected using an enhanced chemiluminescence (ECL) system (Pierce Biotechnology, IL, USA).

MicroRNA Microarray Analysis.

Total RNA was extracted from SKM-1 and LB100-treated-SKM-1 cells using the RNeasy mini kit (Qiagen, Calif., USA) according to the manufacturer's instructions. The miRNA microarray analysis was done by KangChen (Shanghai, China).

RNA Extraction and Quantitation of miR-181b-1 by Real-Time Quantitative RT-PCR.

Total miRNA was extracted from 1×106 SKM-1 cells using the RNAiso kit for small RNA (TaKaRa, Japan), and reverse-transcribed using the One Step PrimeScript miRNA cDNA Synthesis Kit (TaKaRa, Japan). The resulting cDNA was quantified using the iCycler Real-time PCR Detection System (BioRad, Calif., USA) and SYBR Green (Takara, Japan). The expression of miR-181b-1 was quantified relative to the expression of human U6 small nuclear RNA using the 2-°° c.′ method. Primers are listed in Table 1.

In Vivo Tumorigenicity Assays.

All animal studies were performed according to the guidelines of Animal Care and Use Committee of the First Affiliated Hospital of Zhejiang University and met the NIH guidelines for the care and use of laboratory animals. And all animal studies were approved by IACUC committee of the First Affiliated Hospital of Zhejiang University. Nonobese diabetic/severe combined immunodeficiency (NOD/SCID) mice aged 6 weeks were purchased from the Shanghai Experimental Animal Center of the Chinese Academy of Sciences (Shanghai, China). SKM-1 cells (5×106 in 100 μl PBS) were injected subcutaneously into the right flank of each mouse. By 10-14d, when the tumor volumes had reached 90-110 mm3, the mice were randomly divided into four groups: DNR LB100, DNR only, LB100 only, and control. Mice were injected intraperitoneally (i.p.) with 2 mg/kg DNR and/or 2 mg/kg LB100, every other day for a total of 14d. Control mice were injected with an equal volume of PBS. Tumor size was monitored every 3d based on caliper measurements of the two perpendicular diameters; tumor volume was calculated using the formula V=(width′×length×it/6).

Immunohistochemistry Staining.

Immunohistochemical staining was performed on the paraffin-embedded sections. Tissue sections were dewaxed and rehydrated before performing antigen retrieval. Anti-BCL-2 (Cell Signaling Technology, MA, USA) were applied at 1:100 dilution in PBS to incubate slides overnight at 4° C., and incubated with an HRP-conjugated secondary antibody for 1 hat room temperature. DAB was used for color development, and dark brown staining was considered positive. All slides were photographed with optical microscopy Olympus BX51.

TABLE 1
The oligonucleotide sequences used in the study.

| Name | Sequence (5′->3′) |
|---|---|
| miR-181-b-1 F | AACATTCATTGCTGTCGGTGGGT |
| (SEQ ID NO: 1) | |
| U6 F (SEQ ID NO: 2) | TGCGGGTGCTCGCTTCGGCAGC |
| miR-181b-1 precursor | AATCTCGAGGAACCACAGCTTCCT |
| F (SEQ ID NO: 3) | |
| miR-181b-1 precursor | TCCGAATTCACTCCATGTTAGAAC |
| R (SEQ ID NO: 4) | |
| mutant miR-181b-1 F | GGTCACAATCAGGGAAAGGGAAAGTCGG |
| (SEQ ID NO: 5) | |
| mutant miR-181b-1 R | CCGACTTTCCCTTTCCCTGATTGTGACC |
| (SEQ ID NO: 6) | |
| Bcl-2 3′UTR F | GGTAACGCGTCATTATCTTGTCACTG |
| (SEQ ID NO: 7) | |
| Bcl-2 3′UTR R | GGGCAAGCTTCTATTTAACTCTGACC |
| (SEQ ID NO: 8) | |
| Bcl-2 3′UTR mut1 F | ATTAACTTTGCCCGTGACTCTGTTC |
| (SEQ ID NO: 9) | |
| Bcl-2 3′UTR mut1 R | GAACAGAGTCACGGGCAAAGTTAAT |
| (SEQ ID NO: 10) | |
| Bcl-2 3′UTR mut2 F | GTTAGACCGTTGCCCATGATATAAAAG |
| (SEQ ID NO: 11) | |
| Bcl-2 3′UTR mut2 R | CTTTTATATCATGGGCAACGGTCTAAC |
| (SEQ ID NO: 12) | |

F: forward primer; R: reverse primer.

Construction of Retroviral Vectors and Production of Ectopic Retrovirus.

The precursor sequence of miR-181b-1 was PCR amplified from human normal bone marrow mononuclear cells and cloned into MSCVpuro to express miR-181b-1. The mutant miR-181b-1 sequence was created using the primers including the mutated sequences. Primers are listed in Table 1. The MSCVpuro retroviral vector contained a PGK-puromycin-IR ES-GFP (PIG) cassette. The miR-181b-1 precursor sequence or mutant sequence was inserted into the vector between Xhol (CTCGAG) and EcoRI (GAATTC) sites. To produce the ectopic retrovirus, 0.5×106 293T cells were plated in a 60-mm dish the day before transfection. 1.8 μg of retroviral vector DNA and 1.2 μg of PCL-Ampho vector (IMGENEX) were transfected by using the QIAGEN Effectene transfection reagent. Medium was changed with 1 ml of 10% FBS/DMEM after 24 h of transfection. After 48 h of transfection, the virus-containing medium was collected and filtered with a 0.45-μm cellulose acetate (low protein binding) filter.

Dual Luciferase Reporter Assay.

The 3′UTR segment of Bd-2 containing two predicted target sites of miR-181b-1 was inserted into the downstream of the luciferase reporter in the pMIR-REPORT Dual-Luciferase miRNA Target Expression vector. The mutations were constructed using the primers including the mutated sequences. Primers are listed in Table 1. The pMIR-REPORT vector, pRL-TK vector, and miR-181b-1 retrovirus (or scramble control or mutant miR-181b-1 retrovirus) were co-transfected into 293T cells using the QIAGEN Effectene transfection reagent in 24-well plate. The plasmid pRL-TK containing Renilla luciferase was used as internal control. The 293T cells were harvested after infection for 48 h. The relative luciferase activity was measured by the Dual Luciferase Assay System (Promega, Wis., USA).

Statistical Analysis.

All data analyses were performed using GraphPad Prism software version 5.0 (GraphPad, Calif., USA), and inter-group results were assessed for significance using Student's t-test. A two-tailed value of P<0.05 was defined as the threshold of significance.

### Example 1—LB100 Attenuation of PP2A Activity and Reduction of sAML Cell Viability

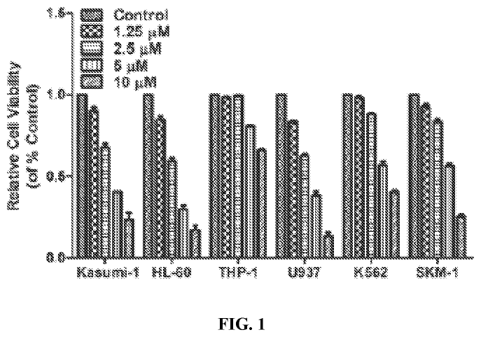

To examine LB100 cytotoxicity, cell viability in 6 different leukemia cell lines including the sAML cell line: SKM-1 was evaluated. Each cell line was determined using an MTT cytotoxicity assay where a linear concentration-dependent cytotoxicity plot for LB100 was seen in all tested cell lines. FIG. 1 shows IC₅₀ values for Kasumi-1, HL-60, THP-1, U937, K562 and SKM-1 at 4 h after treatment were 4.38, 3.36, 13.46, 3.44, 7.00 and 5.35, respectively.

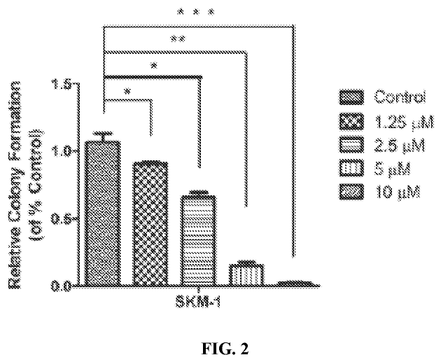

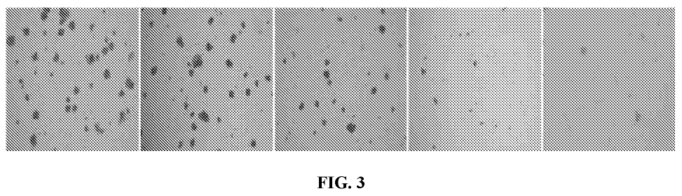

Surprisingly, LB100 exhibited profound cytotoxic activity not only in AML cell lines, but also in the sAML cell line. The dose-dependent inhibitory activity of LB100 on the growth of SKM-1 cells was further confirmed by colony formation assays, as shown in FIGS. 2 and 3.

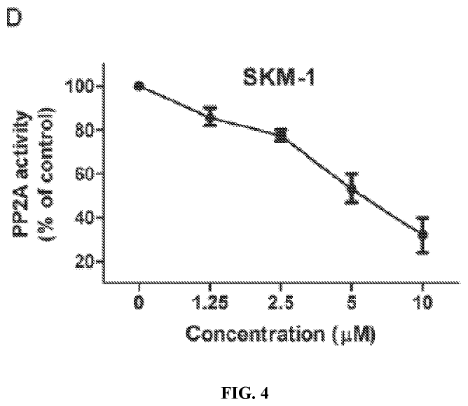

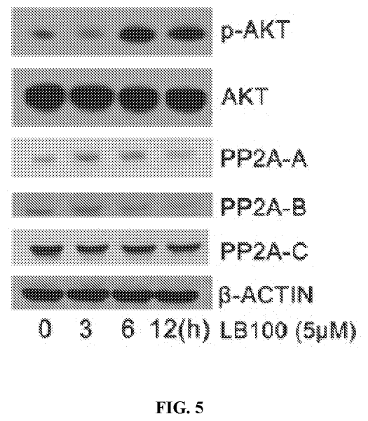

Previous studies have shown that LB100 can reduce PP2A activity in several kinds of solid tumors^{52,57}. Consistent with these findings, exposure to 10 μM LB100 for 12 hours reduced the activity of PP2A up to 60% in SKM-1 cells, as shown in FIG. 4. Moreover, LB100 moderately decreased the expression of the three PP2A subunits (PP2A-A, PP2A-B, and PP2A-C) in sAML cells, as confirmed by western blot method, as shown in FIG. 5. LB100 also increased levels of p-AKT, which is expected since AKT is a direct substrate of PP2A. These results confirm that LB100 effectively inhibits sAML cell growth possibly through PP2A inhibition.

### Example 2—LB100 Modulation of Cell Cycle Regulatory Proteins and G2/M Phase Arrest in sAML Cells

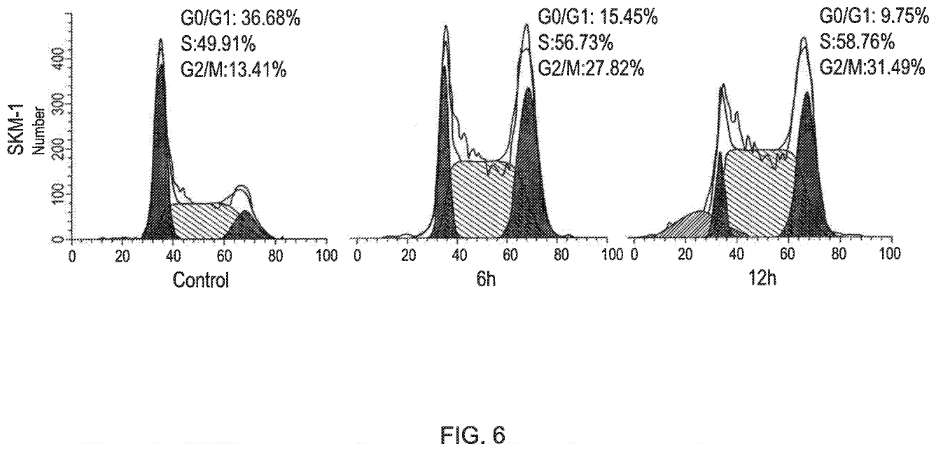

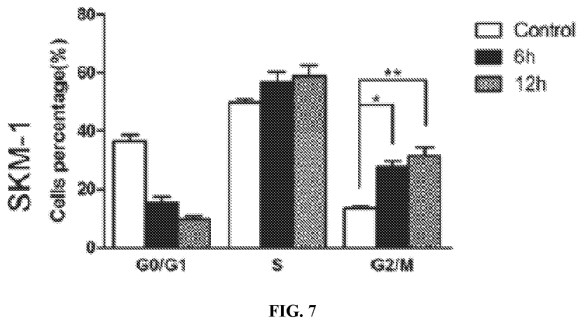

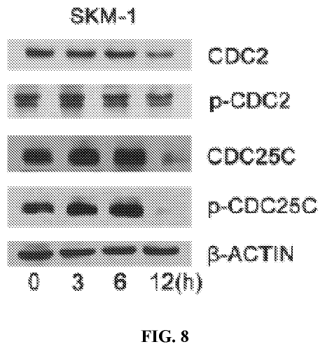

The underlying mechanism for LB100-mediated tumor suppression was further investigated through analysis of changes in cell-cycle behavior and protein expression. Flow analysis of SKM-1 cells demonstrated that 12 h exposure to LB100 at 5 μM dramatically decreased G0/G1 (from 36.7% to 9.8%), and significantly increased G2/M phase cells (from 13.4% to 31.5%), as shown in FIGS. 6 and 7. The accumulation of G2/M phase cells occurred in a time-dependent manner. Consistent with these findings, the G2-to-M checkpoint molecules CDCl₂ and CDCl₂₅C were markedly downregulated in terms of both total- and phosphorylated-protein levels, as shown in FIG. 8. This is in agreement with previous studies investigating LB100 function^{52,54,60}. These findings suggest that LB100 attenuated sAML cell growth at least partly from induction of mitotic cell arrest.

### Example 3—LB100 Induces Apoptotic Cell Death in sAML Cells

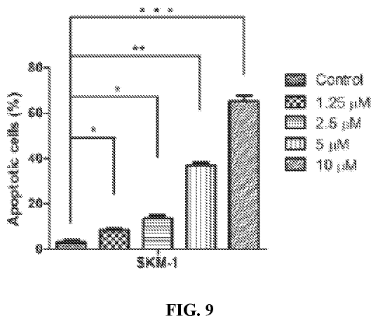

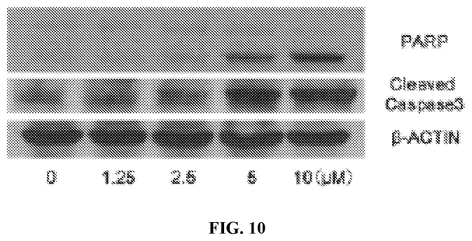

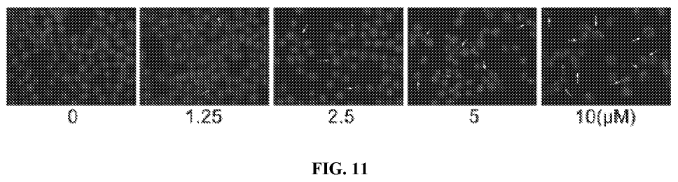

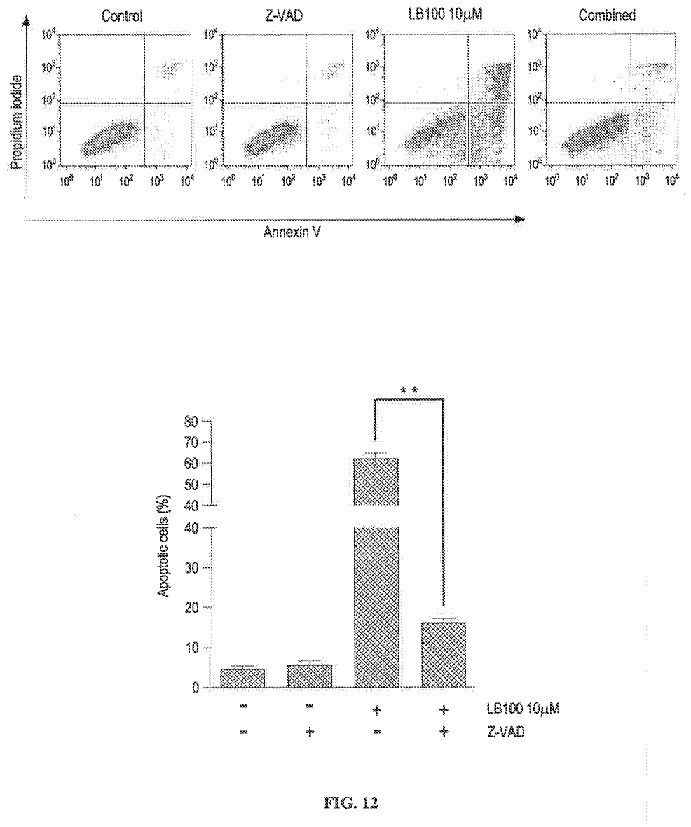

To determine the influence of apoptosis on the observed decreases in cell proliferation after LB100 administration, an Annexin V and Propidium Iodide labeled flow-cytometry assay was used. LB100 demonstrated a concentration-dependent increase in the fraction of apoptotic sAML cells from 3.13% in the absence of LB100, to 8.51%, 13.61%, 37%, and 65.27% in the presence of 1.25 μM, 2.5 μM, 5 μM and 10 μM of LB100, respectively, as shown in FIG. 9. This finding was confirmed with microscopic analysis of sAML cells after Hoechst staining identified increased amounts of condensed, pyknotic nudei, as shown in FIG. 11. Immunoblotting also demonstrated LB100-induced caspase-3 and PARP cleavage in a concentration-dependent manner, as shown in FIG. 10. The effect of pan-caspase inhibition using z-VAD-FMK on LB 100-induced apoptosis was also studied. The inhibitor partially blocked LB100-induced apoptosis, decreasing the rate of apoptosis from 62% to 16%, as shown in FIG. 12. Collectively, these findings suggest that LB100 decreased sAML proliferation at least partly from inducing cellular apoptosis.

### Example 4—LB100 Augments Daunorubicin-Mediated Tumoricidal Effects

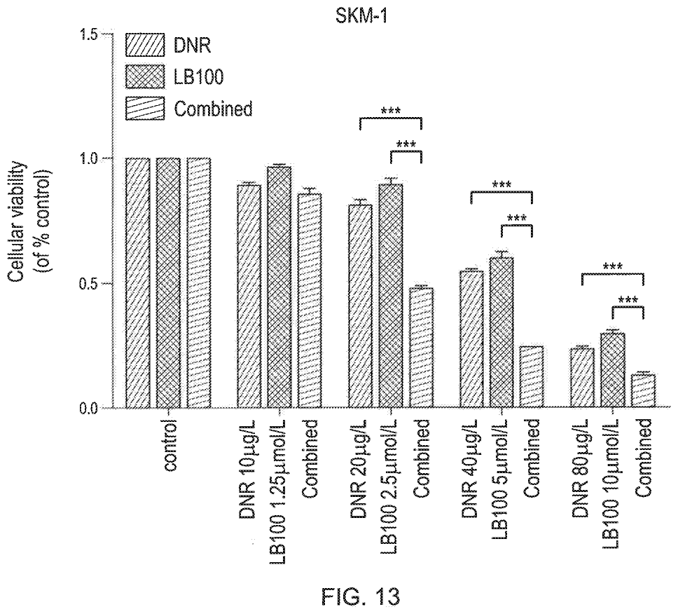

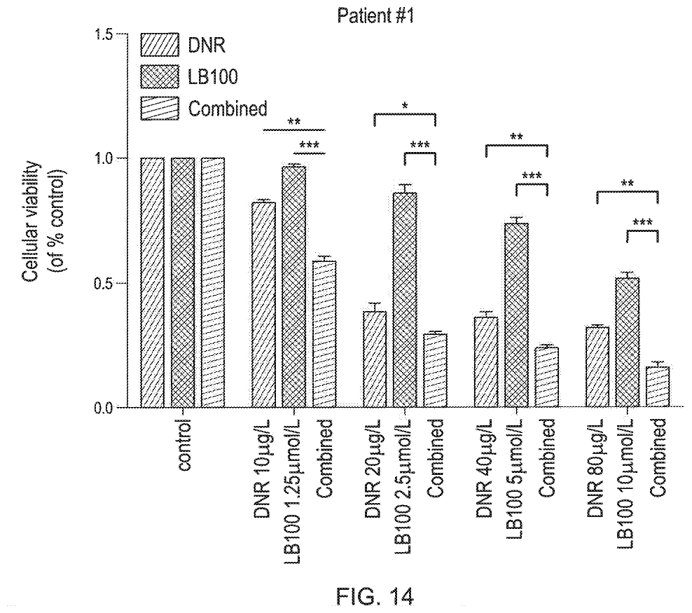

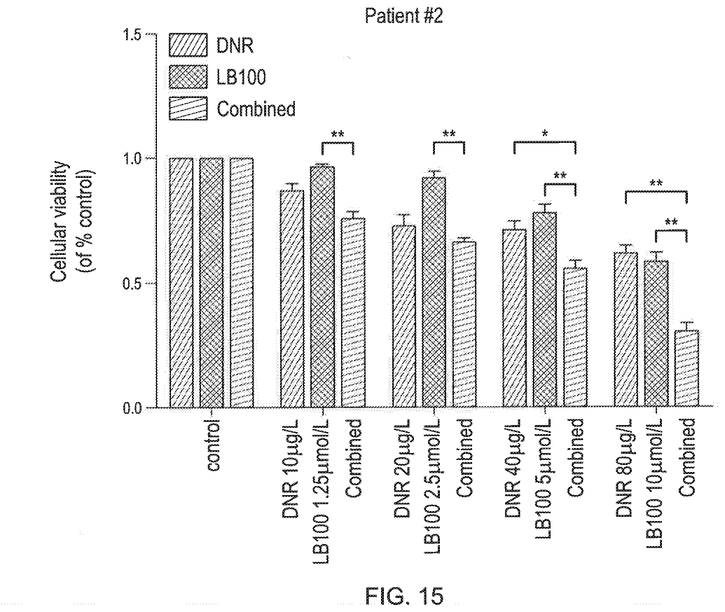

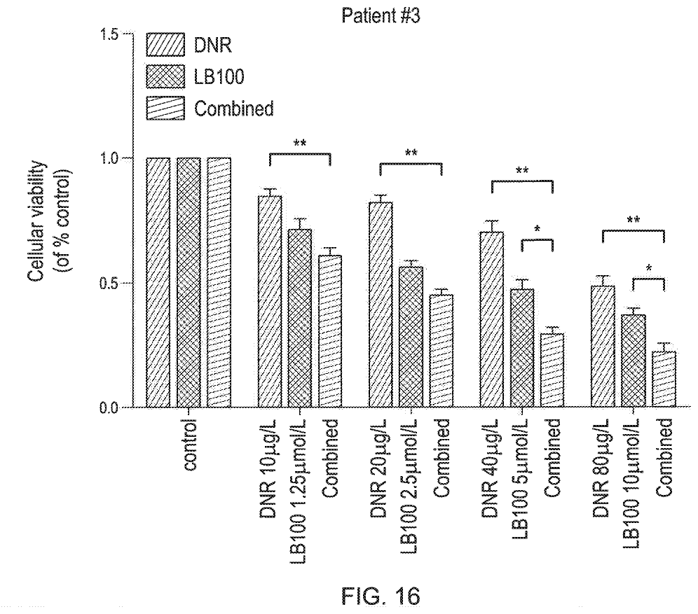

The chemosensitization potential of LB100 was studied using an in-vitro and in-vivo approach to determine whether the tumoricidal effects of daunorubicin (DNR), a common therapeutic agent used in patients with sAML, could be synergistically increased by combinatorial treatment. SKM-1 cell viability was significantly diminished in a dose-dependent manner following 24h incubation with either LB100 or DNR. Simultaneous treatment with LB100 and DNR dramatically reduced SKM-1 cell viability compared to monotherapy with either agent, as shown in FIG. 13. The addition of LB100 similarly sensitized sAML patients' bone marrow mononuclear cells to DNR treatment, as shown in FIGS. 14, 15 and 16. A one- to five-fold increase in the AML suppression ratio was seen in each patient cohort with LB100 and DNR co-treatment.

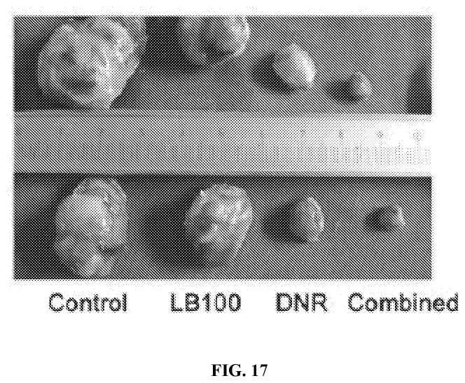

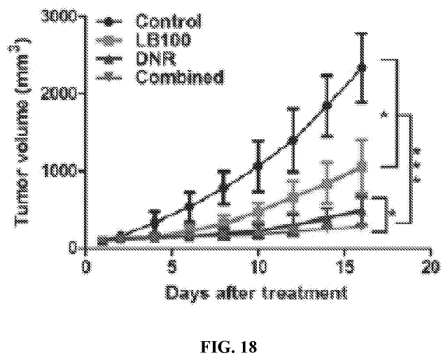

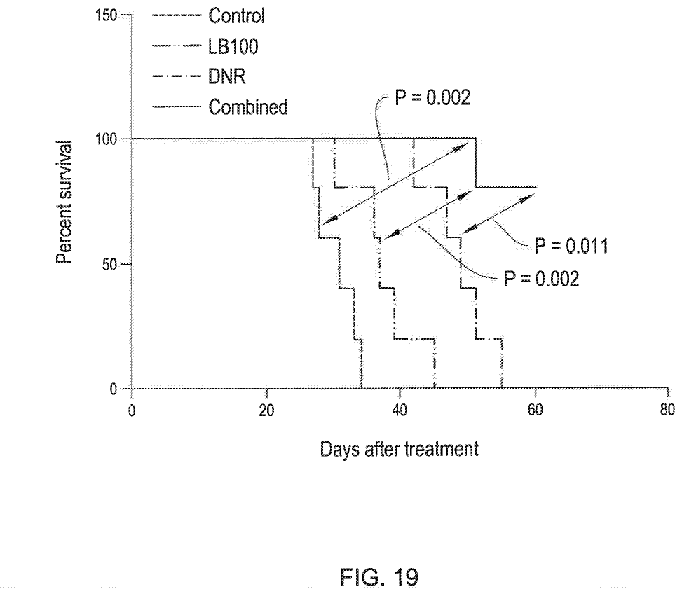

SKM-1 xenografts demonstrated a sharp decrease in tumor volume after receiving either LB100 (P=0.017) or DNR monotherapy (P<0.001) alone as compared to control. Mice receiving the combination therapy of LB100 plus DNR had further decreases in tumor volume as compared with control or either monotherapy, as shown in FIGS. 17 and 18 (control Pc 0.001, LB100 P<0.001, DNR P<0.05). Mice in the combination treatment group were also found to have a significantly prolonged overall survival, as shown in FIG. 19 (control P=0.002, vs LB100 P=0.002, vs DNR P=0.011). These findings demonstrate a synergistic tumoricidal effect with concurrent LB100 and DNR administration. This is in agreement with other studies reporting simultaneous PP2A inhibition enhancing the efficacy of chemotherapy treatment for solid tumors^{31,32}.

### Example 5—LB100 Facilitates sAML Chemosensitivity Through miR-181b-1 Upregulation

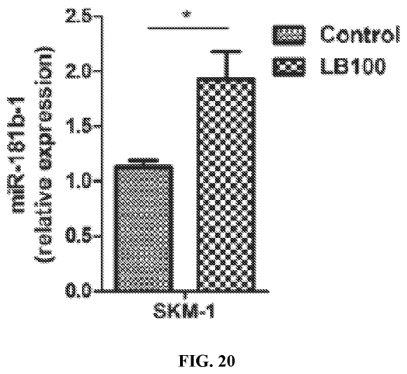

The mechanism underlying LB100 chemosensitization was investigated by assessing the epigenetic response of sAML to LB100 administration. miRNAs are endogenous 17-25 base pair noncoding RNA molecules that play prominent regulatory roles in malignant transformation, stem cell maintenance, metastasis, and invasiveness⁶¹⁻⁶⁷. MicroRNA profiling was performed to analyze the SKM-1 transcriptome for differences after LB100 administration (5 exposure 12h). miR-181b-1 was found to be significantly up-regulated in the LB100 treatment group. Interestingly, miR-181b-1 has previously been identified as an important mediator of cisplatin and vincristine chemosensitivity in human gastric and lung cancer cell lines⁶⁸ qRT-PCR was then performed to confirm upregulation of miR-181b-1 between the LB100 treatment and control group. After normalization to an endogenous control (U6 RNA), the relative expression of miR-181b-1 was found to be increased about 2 fold after LB100 treatment (P=0.049), as shown in FIG. 20.

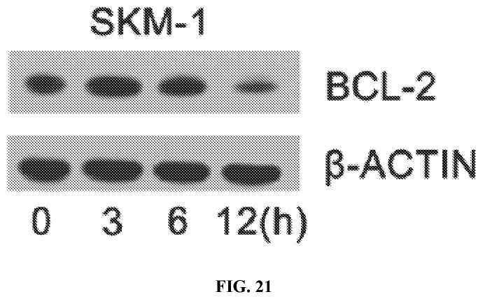

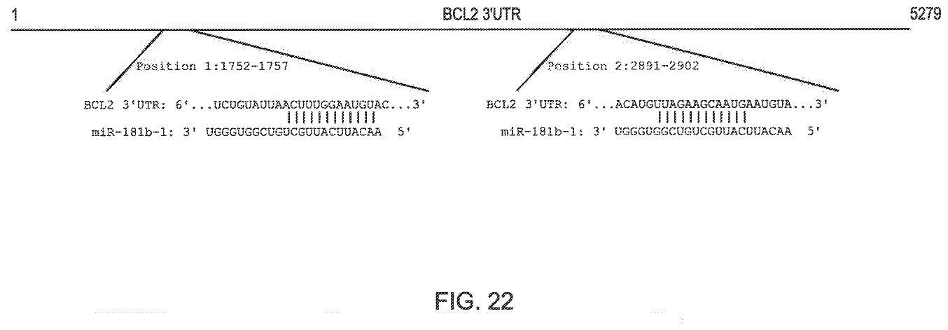

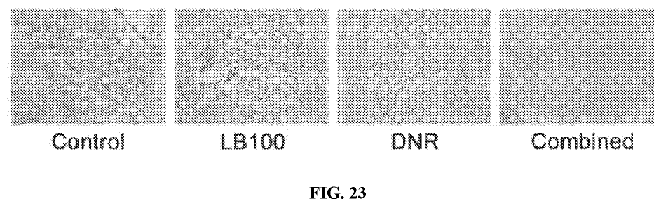

To identify putative targets of miR-181b-1, the online miRNA prediction software TargetScan was utilized to screen transcripts with a 3′ untranslated region (UTR) containing a similar sequence complementarity as miR-181b-1. The Bcl-2 mRNA transcript was identified among the potential targets with a 3′UTR containing two highly conserved 8-mer sites complementary to the seed region of the miR-181b-1, as shown in FIG. 22. Considering the well-characterized anti-apoptotic function of Bcl-2, miR-181b-1 may play an important role in the chemosensitization potential of LB100 by facilitating cell death through inhibition of Bcl-2 translation. Bcl-2 expression was analyzed via immunoblot and immunohistochemistry in in-vitro and in-vivo models, respectively, and was found to be markedly downregulated after LB100 administration, as shown in FIGS. 21 and 23.

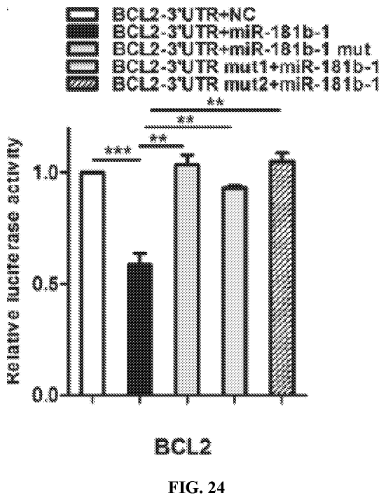

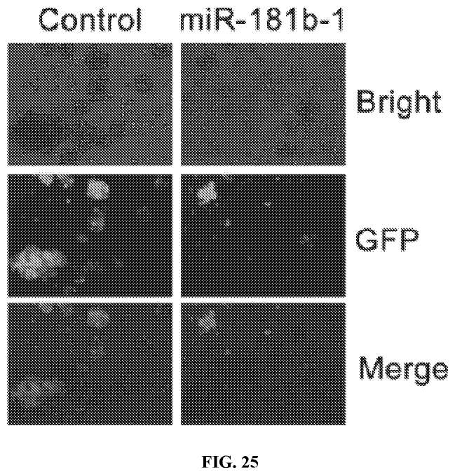

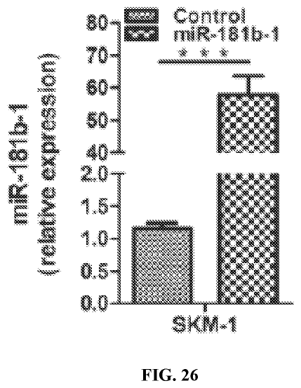

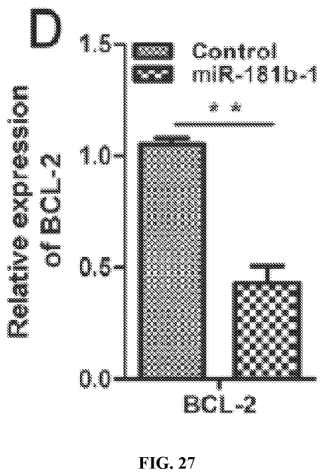

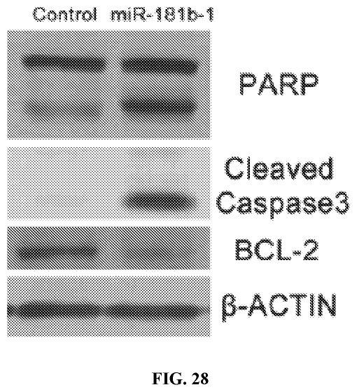

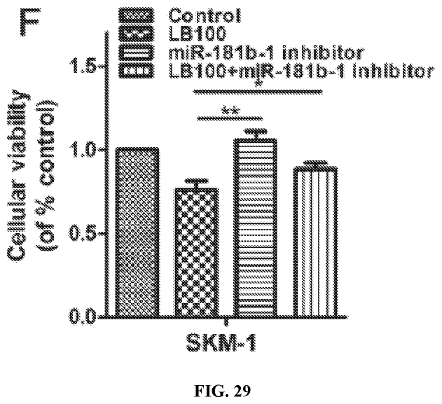

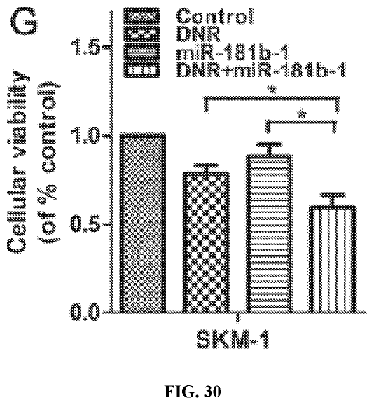

Dual luciferase assays were utilized to confirm whether miR-181b-1 directly interacted with the 3′UTR of the Bcl-2 mRNA transcript. The 3′UTR of Bd-2 was cloned downstream of firefly luciferase using a pMIR-REPORT vector. Normal control (empty vector), miR-181b-1, mutant miR-181b-1, and 3′UTR of Bd-2 binding sites mutant vectors were also utilized. Significant suppression of luciferase activity by miR-181b-1 was observed, as shown in FIG. 24, which was not seen in the other groups. Ectopic miR-181b-1 overexpression greatly decreased Bcl-2 mRNA and protein levels in SKM-1 cells, as shown in FIGS. 25, 26, 27 and 28. Additionally, overexpression of miR-181b-1 to mimic the function of LB100 by activating the caspase cascade, inhibiting cell proliferation, and enhancing DNR cytotoxicity was observed, as shown in FIG. 30. Administration of anti-miRNA specific to miR-181b-1 to SKM-1 cells exposed to LB100 significantly reversed the degree of cell death due to LB100, as shown in FIG. 29. These results suggest that LB100 sensitizes sAML cells to DNR therapy by inducing miR-181b-1 upregulation, causing a subsequent downregulation of Bcl-2.

Patients with myelodysplastic syndromes that secondarily evolve into acute myelogenous leukemia have a median survival of only 15 months despite best standard of care treatment⁵. sAML is characteristically resistant to aggressive induction/consolidation chemotherapy regimens including concomitant cytarabine+daunorubicin. A major mechanism of oncogenic chemoresistance involves overexpression of aberrant anti-apoptotic proteins such as Bcl-2⁶⁹⁻⁷². Indeed, overexpression of Bcl-2 has been shown to accelerate tumorigenesis in transgenic mice, and is notably overexpressed in various diseases including malignant hematonosis^{72,73}. Bcl-2 is an essential intracellular protein that prevents apoptosis by controlling mitochondrial membrane permeability, preventing the release of pro-apoptotic mitochondrial factors such as cytochrome c, halting induction of downstream caspases, and maintaining mitochondrial function⁷⁴⁻⁷⁷. Its overexpression results in an inability of the intrinsic apoptotic pathway to mediate cell death, rendering a distinct survival advantage to mutagenized Downregulation of the Bd-2 oncoprotein can restore the apoptotic pathway and resensitize malignant cells to the effects of therapy-induced apoptosis. Recent studies have reported reversal of chemoresistance using an antisense approach to target Bd-2 in models of chronic lymphocytic leukemia, non-Hodgkin's lymphoma, and multiple myeloma⁷⁹⁻⁸². However, few studies have similarly investigated methods to overcome chemoresistance in models of AML and sAML. Thus, the chemosensitizing potential of LB100, a small-molecule inhibitor of PP2A, in a preclinical model of AML and sAML was examined.

It was found that LB100 suppressed AML and sAML cell proliferation, and enhanced the chemotherapeutic efficacy of daunorubicin (DNR) in sAML cells by halting the cell cycle and facilitating apoptosis. These effects were observed across multiple cell lines and were recapitulated in a mouse sAML xenograft. To determine the mechanism for chemosensitization, the epigenetic response of the sAML cell line to LB100 treatment was explored. MicroRNAs (miRNAs) have been recently suggested as endogenous master regulators of protein expression⁸³. Their differential expression is seen in various disease states, and has the potential to cause or propagate patho-physiologic cell processes^{84,85}. They have been implicated in providing malignant cells their chemoresistant abilities″, and are aberrantly expressed in various subtypes of AML⁹⁰⁻⁹³. miR-181b-1 was identified as significantly upregulated in sAML cells after treatment with LB100. Increased levels of miR-181b-1 have been correlated with improved overall survival in patients with cytogenetically normal, and cytogenetically abnormal AML^{91,94-96}. Recently, Lu et al. showed that miR-181b-1 was downregulated in the chemoresistant human leukemia cell lines K562/A02 and HL60/ADM compared to parental K562 and HL-60 cells⁹⁷. Restoration of miR-181b-1 was noted to sensitize K562/A02 and HL-60/Ara-C cell lines to doxorubicin and cytarabine by targeting HMGB1. To determine the function of miR-181b-1 in sAML, an in-silico analysis using TargetScan to search for miR-181b-1 putative targets was performed based on the complementary 3′ UTR base pair sequence. Bd-2 was identified as a top prospective target based on sequence complementarity. Consistent with the microarray results, a marked increase in miR-181b-1 levels via qRT-PCR in SKM-1 cells treated with LB100 was found. Bcl-2 levels were correspondingly downregulated in the SKM-1 cell line, as well as in an SKM-1 NOD-SCID mouse xenograft. A gain-of-function study was conducted in the sAML cell line through transfection with a retrovirus causing over-expression of miR-181b-1. Dual luciferase assay demonstrated miR-181b-1 directly interacting with the Bcl-2 transcript's 3′ UM, with qRT-PCR and immunoblotting demonstrating an associated decrease in Bd-2 mRNA and protein expression. Furthermore, administration of anti-miR-181b-1 rescued sAML cells from LB100's cytotoxic effects. The effects of miR-181b-1 overexpression in the setting of concurrent DNR administration was investigated and a significant augmentation of DNR sAML cytolytic activity was observed. This is in line with prior results demonstrating LB100-mediated chemosensitivity of DNR to sAML cells. Taken together, these findings suggest that LB100 suppresses sAML cell proliferation, and sensitizes sAML cells to DNR chemotherapy at least partly due to upregulation of miR-181b-1, which in turn downregulates Bcl-2 through direct translational inhibition. Without wishing to be bound by any theory, it is believed to be a novel mechanism of how LB100 augments sAML cell chemosensitivity.

The exact manner underlying how LB100 induces upregulation of miR-181b-1 is still yet to be discovered. In general, the mechanisms behind proteomic regulation of epigenetic molecules are relatively unknown. One report identified a feedback loop involving PP2A, AKT, MYC, and miR-29a, wherein the PP2A substrate MYC was shown to directly suppress miR-29a in a model of AML⁹⁸. Another study demonstrated that knockdown of the PP2A substrate eukaryotic initiation factor 4E (eIF4E) caused a dramatic decrease in miR-134, miR-199b, and miR-424 expression in a model of melanoma⁹⁹. Conversely, eIF4E overexpression had the opposite effect of increasing the expression of the mentioned miRNAs. It is possible that stimulating G2/M cell arrest through inhibition of PP2A leads to upregulation of miR-181b-1 to promote apoptosis of cells through downregulation of Bd-2. The abnormal microtubule configuration at the metaphase plate in cells given selective PP2A inhibitors¹⁰⁰, might serve as a distress signal indicating the non-viability of the cell. Further experiments are needed to investigate the sequential actions involving LB100 upregulation of miR-181b-1.

The dephosphorylation of CDCl2 and CDCl25C after selective PP2A inhibition has previously been noted by other investigators¹⁰¹. Prior to the onset of mitosis, there is a highly regulated balance between cyclinB-Cdc2 and PP2A¹⁰²⁻¹⁰⁴. The balance dictates the phosphorylation level of mitotic substrates, including CDCl2 and CDCl25C, and is essential to allow the correct entry into and exit from mitosis¹⁰⁵⁻¹⁰⁷. At baseline, PP2A is held in a state of incomplete activation by its regulator Greatwall¹⁰¹. LB100 administration results in a dose- and time-dependent inactivation of PP2A (FIGS. 4 and 5), resulting in the time-dependent dephosphorylation of CDCl2 and CDCl25C (FIG. 8). It is unknown why CDCl2 and CDCl25C are degraded with LB100, however other studies have seen similar results with PP2A-specific inhibition¹⁰¹. The ubiquitin proteosomal system is intimately associated with these mitotic substrates, and it is possible that alterations of their phosphorylation status might promote their ubiquitin-dependent degradation. These findings are in line with previous results demonstrating G2-M cell cycle arrest after selective PP2A inhibition³².

PP2A is a complex molecule that is often targeted for activation in models of malignancy due to its occasional tumor-suppressive properties. FTY720 is a PP2A activator that has shown promising results in preclinical models of AML. To explain this finding, differences in baseline expression of PP2A need to be accounted. Cell lines most responsive to FTY720 have a specific D816V mutation in the tyrosine kinase domain of c-kit¹⁰⁸⁻¹⁰⁹. This mutation causes decreased basal expression of PP2A, reduced PP2A activity, and higher baseline activation of the Ras/Raf/MEK/ERK signaling cascade¹⁰⁹. Increased activation of the Ras/Raf/MEK/ERK signaling pathway is known to be associated with malignant transformation of pre-cancerous cells¹¹⁰. Administration of FTY720 to AML cells with the D816V mutation is associated with decreased expression of Ras/Raf/MEK/ERK, and decreased cell viability¹⁰⁹. The AML/sAML cell lines utilized differed in that they had low baseline expression of the Ras/Raf/MEK/ERK pathways, along with relatively higher levels of PP2A. These studies demonstrated evidence of pro-apoptotic processes after LB100 administration such as decreased Bd-2 expression, increased cleaved caspase 3 levels (FIGS. 10 and 21), and increased phosphorylated Bcl-2 and CamKII. PP2A is known to promote resistance to apoptosis through dephosphorylative activation of CaMKII¹⁸. The phosphorylation of Bcl-2 can manifest as a pro-apoptotic signal^{111,112}. And PP2A is known to inhibit apoptosis by dephosphorylating Bd-2 in various tumor cell lines¹⁷. Annexin V and propidium iodide FACS analysis (FIGS. 9 and 12) were utilized to demonstrate increased apoptosis after PP2A inhibition in sAML cells. Interestingly, increased activation of the anti-apoptotic Ras/Raf/MEK/ERK signaling cascade in the same sAML cell line was observed. This interesting finding is in accordance with previous investigation involving transformed mesenchymal stem cells (rTDMCs) in a model of aggressive sarcoma³². As with many types of AML and sAML, the rTDMC cell line does not demonstrate a baseline inhibition of PP2A. The intrinsic differences in baseline oncogenic signaling pathways might explain the differences in susceptibility to PP2A inhibition vs activation.

In summary, LB100 has therapeutic potential in the treatment of sAML. As a monotherapy it evokes apoptosis and cell cycle arrest in sAML cells. It synergizes with DNR to provide enhanced sAML cytotoxicity. Evidence that LB100 induces upregulation of miR-181b-1 to suppress the proapoptotic protein Bcl-2 has been observed. These findings provide preclinical support for testing LB100 as an adjunct to DNR to overcome sAML multi-drug resistance.

### ENUMERATED EMBODIMENTS

In a first embodiment, the invention is a method for treating secondary acute myeloid leukemia (sAML) in a patient comprising administering a PP2A inhibitor having the structure:

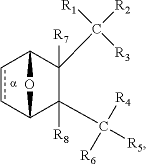

wherein:
bond α is present or absent;
R₁ and R₂ together are ═O;
R₃ is OH, O⁻, OR₉, O(CH₂)₁₋₆R₉, SH, S⁻, or SR₉, wherein R₉ is H, alkyl, alkenyl, alkynyl or aryl;
R₄ is

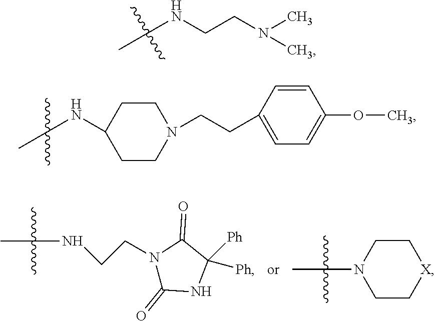

where X is O, S, NR₁₀, or N⁺R₁₀R₁₀, where each R₁₀ is independently H, alkyl, alkenyl, alkynyl, aryl,

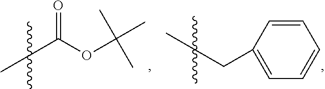

—CH₂CN, —CH₂CO₂R₁₁, or —CH₂COR₁₁, wherein each R₁₁ is independently H, alkyl, alkenyl or alkynyl;
R₅ and R₆ taken together are ═O;
R₇ and R₈ are each H,
or a salt, zwitterion, or ester thereof.

In a second embodiment, the invention is a method according to the first embodiment, wherein the PP2A inhibitor has the structure:

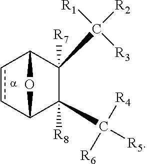

In a third embodiment, the invention is a method according to the first or second embodiment, wherein bond α is absent.

In a fourth embodiment, the invention is a method according to the first or second embodiment, wherein bond α is present.

In a fifth embodiment, the invention is a method according to the first-third embodiments, wherein the PP2A inhibitor has the structure:

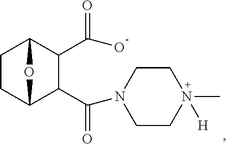

or a salt or ester thereof.

In a sixth embodiment, the invention is a method according to the first-fifth embodiments, further comprising administration of an anti-cancer agent.

In a seventh embodiment, the invention is a method according to the sixth embodiment, wherein the anti-cancer agent is daunorubicin.

In an eighth embodiment, the invention is a method according to the fifth or sixth embodiment, wherein the administration of the PP2A inhibitor enhances cytotoxicity of the anti-cancer agent.

In a ninth embodiment, the invention is a method according to the eighth embodiment, wherein the administration of the PP2A inhibitor enhances cytotoxicity of the anti-cancer agent via upregulation of miR-181b-1.

In a tenth embodiment, the invention is a method for treating secondary acute myeloid leukemia (sAML) in a patient comprising administering a PP2A inhibitor in combination with an anti-cancer agent so as to thereby treat sAML; wherein the PP2a inhibitor has the structure:

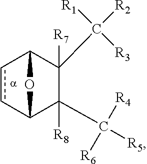

wherein:
bond α is present or absent;
R₁ and R₂ together are ═O;
R₃ is OH, O⁻, OR₉, O(CH₂)₁₋₆R₉, SH, S⁻, or SR₉, wherein R₉ is H, alkyl, alkenyl, alkynyl or aryl;
R₄ is

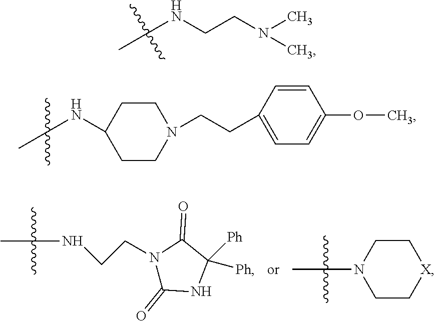

where X is O, S, NR₁₀, N⁺HR₁₀ or N⁺R₁₀R₁₀, where each R₁₀ is independently H, alkyl, alkenyl, alkynyl, aryl,

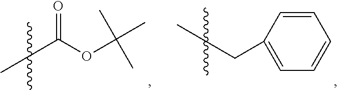

—CH₂CN, —CH₂CO₂R₁₁, or —CH₂COR₁₁, wherein each R₁₁ is independently H, alkyl, alkenyl or alkynyl;
R₅ and R₆ taken together are ═O;
R₇ and R₈ are each H,
or a salt, zwitterion, or ester thereof.

In an eleventh embodiment, the invention is a method according to the tenth embodiment, wherein the PP2A inhibitor has the structure:

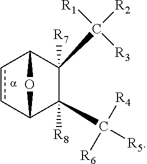

In a twelfth embodiment, the invention is a method according to the tenth or eleventh embodiment, wherein bond α is absent.

In a thirteenth embodiment, the invention is a method according to the tenth or eleventh embodiment, wherein bond α is present.

In a fourteenth embodiment, the invention is a method according to the tenth-twelfth embodiments, wherein the PP2A inhibitor has the structure:

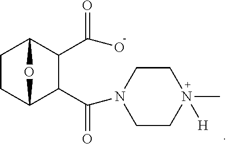

or a salt or ester thereof.

In a fifthteenth embodiment, the invention is a method according to the tenth-fourteenth embodiments, wherein the anti-cancer agent is daunorubicin.

In a sixteenth embodiment, the invention is a method according to the tenth-fifthteenth embodiments, wherein the administration of the PP2A inhibitor enhances cytotoxicity of the anti-cancer agent.

In a seventeenth embodiment, the invention is a method according to the tenth-sixteenth embodiments, wherein the administration of the PP2A inhibitor enhances cytotoxicity of the anti-cancer agent via upregulation of miR-181b-1.

In an eighteenth embodiment, the invention is a method according to the tenth-seventeenth embodiments, wherein the PP2A inhibitor and the anti-cancer agent are administered simultaneously, separately or sequentially.

In a nineteenth embodiment, the invention is a method of enhancing cytotoxicity of an anti-cancer agent in a patient afflicted with sAML comprising administering to the patient a PP2A inhibitor having the structure:

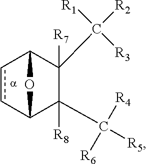

wherein:
bond α is present or absent;
R₁ and R₂ together are ═O;
R₃ is OH, O⁻, OR₉, O(CH₂)₁₋₆R₉, SH, S⁻, or SR₉, wherein R₉ is H, alkyl, alkenyl, alkynyl or aryl;
R₄ is

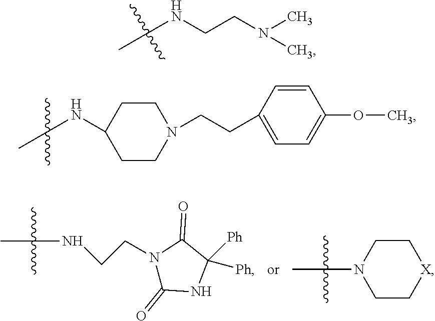

where X is O, S, NR₁₀, N⁺HR₁₀ or N⁺R₁₀R₁₀, where each R₁₀ is independently H, alkyl, alkenyl, alkynyl, aryl,

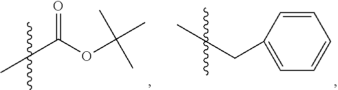

—CH₂CN, —CH₂CO₂R₁₁, or —CH₂COR₁₁, wherein each R₁₁ is independently H, alkyl, alkenyl or alkynyl;
R₅ and R₆ taken together are ═O;
R₇ and R₈ are each H,
or a salt, zwitterion, or ester thereof.

In an twentieth embodiment, the invention is a method according to the nineteenth embodiment, wherein the PP2A inhibitor has the structure:

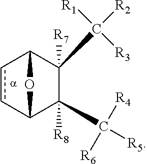

In a twenty-first embodiment, the invention is a method according to the nineteenth or twentieth embodiment, wherein bond α is absent.

In a twenty-second embodiment, the invention is a method according to the nineteenth or twentieth embodiment, wherein bond α is present.

In a twenty-third embodiment, the invention is a method according to the nineteenth-twenty-first embodiments, wherein the PP2A inhibitor has the structure:

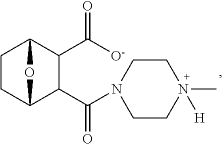

or a salt or ester thereof.

In a twenty-fourth embodiment, the invention is a method according to the nineteenth-twenty-third embodiments, wherein the anti-cancer agent is daunorubicin.

In a twenty-fifth embodiment, the invention is a method according to the nineteenth-twenty-fourth embodiments, wherein the administration of the PP2A inhibitor enhances cytotoxicity of the anti-cancer agent via upregulation of miR-181b-1.

While we have described a number of embodiments of this invention, it is apparent that the basic examples may be altered to provide other embodiments that utilize the compounds and methods of this invention. Therefore, it will be appreciated that the scope of this invention is to be defined by the appended claims rather than by the specific embodiments that have been represented by way of example.

### REFERENCES

1. Tefferi, A. & Vardiman, J. W. Myelodysplastic syndromes. N Engl J Med 361, 1872-1885 (2009).
2. Cogle, C. It, Craig, B. M., Rollison, D. E. & List, A. F. Incidence of the myelodysplastic syndromes using a novel claims-based algorithm: high number of uncaptured cases by cancer registries. Blood 117, 7121-7125 (2011).
3. Vardiman, J. W. The World Health Organization (WHO) classification of tumors of the hematopoietic and lymphoid tissues: an overview with emphasis on the myeloid neoplasms. Chem Biol Interact 184, 16-20 (2010).
4. Walter, M. J. et al. Clonal architecture of secondary acute myeloid leukemia. N Engl J Med 366, 1090-1098 (2012).
5. Shukron, O., Vainstein, V., Kundgen, A., Germing, U. & Agur, Z. Analyzing transformation of myelodysplastic syndrome to secondary acute myeloid leukemia using a large patient database. Ant J Hematol 87, 853-860 (2012).
6. Rajapaksa, It, Ginzton, N., Rott, L S. & Greenberg, P. L. Altered oncoprotein expression and apoptosis in myelodysplastic syndrome marrow cells. Blood 88, 4275-4287 (1996).
7. Boncrary, D. et al. Fas/Apo-1 (CD95) expression and apoptosis in patients with myelodysplastic syndromes. Leukemia 11, 839-845 (1997).
8. Davis, R. E. & Greenberg, P. L. Bcl-2 expression by myeloid precursors in myelodysplastic syndromes: relation to disease progression. Leuk Res 22, 767-777 (1998).
9. Quesnel, B. et al. Methylation of the p15(INK4b) gene in myelodysplastic syndromes is frequent and acquired during disease progression. Blood 91, 2985-2990 (1998).
10. Kirsch, D. G. & Kastan, M. B. Tumor-suppressor p53: implications for tumor development and prognosis. J Clin Oncol 16, 3158-3168 (1998).
11. Simboeck, E. et al. A phosphorylation switch regulates the transcriptional activation of cell cycle regulator p21 by histone deacetylase inhibitors. J Biol Chem 285, 41062-41073 (2010).
12. Bononi, A. et al. Protein kinases and phosphatases in the control of cell fate. Enzyme Res 2011, 329098 (2011).
13. Eichhorn, P. J., Creyghton, M. P. & Bernards, R. Protein phosphatase 2A regulatory subunits and cancer. Biochim Biophys Acta 1795, 1-15 (2009).
14. Parsons, R. Phosphatases and tumorigenesis. Curr Opin Oncol 10, 88-91(1998).
15. Schonthal, A. H. Role of serine/threonine protein phosphatase 2A in cancer. Cancer Lett 170, 1-13 (2001).
16. Francis, G. et al. Identification by differential display of a protein phosphatase-2A regulatory subunit preferentially expressed in malignant melanoma cells. Int J Cancer 82, 709-713 (1999).
17. Simizu, S., Tamura, Y. & Osada, H. Dephosphorylation of Bcl-2 by protein phosphatase 2A results in apoptosis resistance. Cancer Sci 95, 266-270 (2004).
18. Huang, B. et al. Metabolic control of Ca2+/calmodulin-dependent protein kinase II (CaMKII)-mediated caspase-2 suppression by the B55beta/protein phosphatase 2A (PP2A). J Biol Chem 289, 35882-35890 (2014).
19. Ugi, S., Imamura, T., Ricketts, W. & Olefsky, J. M. Protein phosphatase 2A forms a molecular complex with Shc and regulates Shc tyrosine phosphorylation and downstream mitogenic signaling. Mol Cell Biol 22, 2375-2387 (2002).
20. Zhou, B., Wang, Z. X., Zhao, Y., Brautigan, D. L. & Zhang, Z. Y. The specificity of extracellular signal-regulated kinase 2 dephosphorylation by protein phosphatases. J Biol Chem 277, 31818-31825 (2002).
21. Abraham, D. et al. Raf-1-associated protein phosphatase 2A as a positive regulator of kinase activation. J Biol Chem 275, 22300-22304 (2000).
22. Ory, S., Thou, M., Conrads, T. P., Veenstra, T. D. & Morrison, D. K. Protein phosphatase 2A positively regulates Ras signaling by dephosphorylating KSR1 and Raf-1 on critical 14-3-3 binding sites. Curr Biol 13, 1356-1364 (2003).
23. Gendron, S., Couture, J. & Aoudjit, F. Integrin alpha2betal inhibits Fas-mediated apoptosis in T lymphocytes by protein phosphatase 2A-dependent activation of the MAPK/ERK pathway. J Biol Chem 278, 48633-48643 (2003).
24. Ajay, A. K. et al. Cdk5 phosphorylates non-genotoxically overexpressed p53 following inhibition of PP2A to induce cell cycle arrest/apoptosis and inhibits tumor progression. Mol Cancer 9, 204 (2010).
25. Boudreau, R. T., Conrad, D. M. & Hoskin, D. W. Apoptosis induced by protein phosphatase 2A (PP2A) inhibition in T leukemia cells is negatively regulated by PP2A-associated p38 mitogen-activated protein kinase. Cell Signal 19, 139-151 (2007).
26. Benito, A., Lerga, A., Silva, M., Leon, J. & Fernandez-Luna, J. L Apoptosis of human myeloid leukemia cells induced by an inhibitor of protein phosphatases (okadaic acid) is prevented by Bcl-2 and Bcl-X(L). Leukemia 11, 940-944 (1997).
27. Ishida, Y., Furukawa, Y., Decaprio, J. A., Saito, M. & Griffin, J. D. Treatment of myeloid leukemic cells with the phosphatase inhibitor okadaic acid induces cell cycle arrest at either G1/S or G2/M depending on dose. J Cell Physiol 150, 484-492 (1992).
28. Lerga, A. et al. Apoptosis and mitotic arrest are two independent effects of the protein phosphatases inhibitor okadaic acid in K562 leukemia cells. Biochem Biophys Res Commun 260, 256-264 (1999).
29. Riordan, F. A., Foroni, L, Hoffbrand, A. V., Mehta, A. B. & Wickremasinghe, R, G. Okadaic acid-induced apoptosis of HL60 leukemia cells is preceded by destabilization of bd-2 mRNA and downregulation of bd-2 protein. FEBS Lett 435, 195-198 (1998).
30. Sallman, D. A., Wei, S. & List, A. PP2A: The Achilles Heal in MDS with 5q Deletion. Front Oncol 4, 264 (2014).
31. Lu, J. et al. The effect of a PP2A inhibitor on the nuclear receptor corepressor pathway in glioma. J Neurosurg 113, 225-233 (2010).
32. Zhang, C. et al. A synthetic cantharidin analog for the enhancement of doxorubicin suppression of stem cell-derived aggressive sarcoma. Biomaterials 31, 9535-9543 (2010).
33. Bai, X. et al. Inhibition of protein phosphatase 2A sensitizes pancreatic cancer to chemotherapy by increasing drug perfusion via HIF-1alpha-VEGF mediated angiogenesis. Cancer Lett 355, 281-287 (2014).
34. Millward, T. A., Zolnierowicz, S. & Hemmings, B. A. Regulation of protein kinase cascades by protein phosphatase 2A. Trends Biochem Sci 24, 186-191 (1999).
35. Wang, R., Lv, L., Zhao, Y. & Yang, N. Okadaic acid inhibits cell multiplication and induces apoptosis in a549 cells, a human lung adenocarcinoma cell line. Int J Clin Exp Med 7, 2025-2030 (2014).
36. Liu, C. Y. et al. Tamoxifen induces apoptosis through cancerous inhibitor of protein phosphatase 2A-dependent phospho-Akt inactivation in estrogen receptor-negative human breast cancer cells. Breast Cancer Res 16, 431 (2014).
37. Ferron, P. J., Hogeveen, K., Fessard, V. & Le Hegarat, L. Comparative analysis of the cytotoxic effects of okadaic acid-group toxins on human intestinal cell lines. Mar Drugs 12, 4616-4634 (2014).
38. Valdiglesias, V., Prego-Faraldo, M. V., Pasaro, E., Mendez, J. & Laffon, B. Okadaic acid: more than a diarrheic toxin. Mar Drugs 11, 4328-4349 (2013).
39. Kamat, P. K., Rai, S., Swarnkar, S., Shukla, R. & Nath, C. Molecular and cellular mechanism of okadaic acid (OKA)-induced neurotoxicity: a novel tool for Alzheimer's disease therapeutic application. Mol Neurobiol 50, 852-865 (2014).
40. Wang, G. S. Medical uses of mylabris in ancient China and recent studies. J Ethnopharmacol 26, 147-162 (1989).
41. Chen, Y. N. et al. Effector mechanisms of norcantharidin-induced mitotic arrest and apoptosis in human hepatoma cells. Int J Cancer 100, 158-165 (2002).
42. Huan, S. K., Lee, H. H., Liu, D. Z., Wu, C. C. & Wang, C. C. Cantharidin-induced cytotoxicity and cyclooxygenase 2 expression in human bladder carcinoma cell line. Toxicology 223, 136-143 (2006).
43. Williams, L. A., Moller, W., Merisor, E., Kraus, W. & Rosner, H. In vitro anti-proliferation/cytotoxic activity of cantharidin (Spanish Fly) and related derivatives. West Indian Med 152, 10-13 (2003).
44. Li, W. et al. Cantharidin, a potent and selective PP2A inhibitor, induces an oxidative stress-independent growth inhibition of pancreatic cancer cells through G2/M cell-cycle arrest and apoptosis. Cancer Sci 101, 1226-1233 (2010).
45. Wang, C. C., Wu, C. H., Hsieh, K. J., Yen, K. Y. & Yang, L. L. Cytotoxic effects of cantharidin on the growth of normal and carcinoma cells. Toxicology 147, 77-87 (2000).
46. Huang, W. W. et al. Cantharidin induces G2/M phase arrest and apoptosis in human colorectal cancer colo 205 cells through inhibition of CDKI activity and caspase-dependent signaling pathways. Int J Oncol 38, 1067-1073 (2011).
47. Kuo, J. H. et al. Cantharidin induces apoptosis in human bladder cancer TSGH 8301 cells through mitochondria-dependent signal pathways. Int J Oncol 37, 1243-1250 (2010).
48. Li, Y. M. & Casida, J. E. Cantharidin-binding protein: identification as protein phosphatase 2A. Proc Natl Acad Sci USA 89, 11867-11870(1992).
49. Peng, F. et al. Induction of apoptosis by norcantharidin in human colorectal carcinoma cell lines: involvement of the CD95 receptor/ligand. J Cancer Res Clin Oncol 128, 223-230 (2002).
50. Karras, D. J., Farrell, S. E., Harrigan, R. A., Henretig, F. M. & Gealt, L. Poisoning from “Spanish fly” (cantharidin). Am J Emerg Med 14, 478-483 (1996).
51. Sandroni, P. Aphrodisiacs past and present: a historical review. Clin Auton Res 11, 303-307 (2001).
52. Chang, K. E. et al. The protein phosphatase 2A inhibitor LB100 sensitizes ovarian carcinoma cells to cisplatin-mediated cytotoxicity. Mol Cancer Ther 14, 90-100 (2015).
53. Lv, P. et al. Inhibition of protein phosphatase 2A with a small molecule LB100 radiosensitizes nasopharyngeal carcinoma xenografts by inducing mitotic catastrophe and blocking DNA damage repair. Oncotarget 5, 7512-7524 (2014).
54. Lu, J. et al. Inhibition of serine/threonine phosphatase PP2A enhances cancer chemotherapy by blocking DNA damage induced defense mechanisms. Proc Natl Acad Sci USA 106, 11697-11702 (2009).
55. Martiniova, L. et al. Pharmacologic modulation of serine/threonine phosphorylation highly sensitizes PHEO in a MPC cell and mouse model to conventional chemotherapy. PLoS One 6, e14678 (2011).
56. Srivastava, R. K., Kurzrock, R. & Shankar, S. MS-275 sensitizes TRAIL-resistant breast cancer cells, inhibits angiogenesis and metastasis, and reverses epithelial-mesenchymal transition in vivo. Mol Cancer Ther 9, 3254-3266 (2010).
57. Bai, X. L. et al. Inhibition of protein phosphatase 2A enhances cytotoxicity and accessibility of chemotherapeutic drugs to hepatocellular carcinomas. Mol Cancer Ther 13, 2062-2072 (2014).
58. Wei, D. et al. Inhibition of protein phosphatase 2A radiosensitizes pancreatic cancers by modulating CDCl25C/CDKI and homologous recombination repair. Clin Cancer Res 19, 4422-4432 (2013).
59. Chung, V. M. M. A. & Kovach, J. A. phase 1 study of a novel inhibitor of protein phosphatase 2A alone and with docetaxel. J Clin Oncol 32, TS2636 (2014).
60. Chen, X. et al. The microtubule depolymerizing agent CYT997 effectively kills acute myeloid leukemia cells via activation of caspases and inhibition of PI3K/Akt/mTOR pathway proteins. Exp Ther Med 6, 299-304 (2013).
61. Katsushima, K. & Kondo, Y. Non-coding RNAs as epigenetic regulator of glioma stem-like cell differentiation. Front Genet 5, 14 (2014).
62. Chu, P. M. et al. Deregulated microRNAs identified in isolated glioblastoma stem cells: an overview. Cell Transplant 22, 741-753 (2013).
63. Godlewski, J., Newton, H. B., Chiocca, E. A. & Lawler, S. E. MicroRNAs and glioblastoma; the stem cell connection. Cell Death Differ 17, 221-228 (2010).
64. Nikaki, A., Piperi, C. & Papavassiliou, A. G. Role of microRNAs in gliomagenesis: targeting miRNAs in glioblastoma multiforme therapy. Expert Opin Mvestig Drugs 21, 1475-1488 (2012).
65. Peruzzi, P. et al. MicroRNA-128 coordinately targets Polycomb Repressor Complexes in glioma stem cells. Neuro Oncol 15, 1212-1224 (2013).
66. Godlewski, J. et al. MicroRNA-451 regulates LKB1/AMPK signaling and allows adaptation to metabolic stress in glioma cells. Mol Cell 37, 620-632 (2010).
67. Godlewski, J., Bronisz, A., Nowicki, M. O., Chiocca, E. A. & Lawler, S. microRNA-451: A conditional switch controlling glioma cell proliferation and migration. Cell Cycle 9, 2742-2748 (2010).
68. Zhu, W, Shan, X., Wang, T, Shu, Y. & Liu, P. miR-181b modulates multidrug resistance by targeting BCL2 in human cancer cell lines. Int J Cancer 127, 2520-2529 (2010).
69. Schmitt, C. A., Rosenthal, C. T. & Lowe, S. W. Genetic analysis of chemoresistance in primary murine lymphomas. Nat Med 6, 1029-1035(2000).
70. Reed, J. C. Dysregulation of apoptosis in cancer. J Clin Oncol 17, 2941-2953 (1999).
71. Wei, M. C. et al. Proapoptotic BAX and BAK: a requisite gateway to mitochondria] dysfunction and death. Science 292, 727-730 (2001).
72. Zhang, L., Yu, J., Park, B. H., Kinzler, K. W. & Vogelstein, B. Role of BAX in the apoptotic response to anticancer agents. Science 290, 989-992 (2000).
73. Adams, J. M., Harris, A. W, Strasser, A., Ogilvy, S. & Cory, S. Transgenic models of lymphoid neoplasia and development of a pan-hematopoietic vector. Oncogene 18, 5268-5277, doi:10.1038/sj.onc.I202997 (1999).
74. Adams, J. M. & Cory, S. The Bd-2 protein family: arbiters of cell survival. Science 281, 1322-1326 (1998).
75. Martinou, J. C. & Green, D. R. Breaking the mitochondrial barrier. Nat Rev Mol Cell Biol 2, 63-67 (2001).
76. Huang, D. C. & Strasser, A. BH3-Only proteins-essential initiators of apoptotic cell death. Cell 103, 839-842 (2000).
77. Wang, X. The expanding role of mitochondria in apoptosis. Genes Dev 15, 2922-2933 (2001).
78. Johnstone, R. W, Ruefli, A. A. & Lowe, S. W. Apoptosis: a link between cancer genetics and chemotherapy. Cell 108, 153-164 (2002).
79. Marcucci, G. et al. Phase 1 and pharmacodynamic studies of G3139, a Bcl-2 antisense oligonucleotide, in combination with chemotherapy in refractory or relapsed acute leukemia. Blood 101, 425-432 (2003).
80. Pepper, C., Hooper, K., Thomas, A., Hoy, T. & Bentley, P. Bd-2 antisense oligonucleotides enhance the cytotoxicity of chlorambucil in B-cell chronic lymphocytic leukemia cells. Leuk Lymphoma 42, 491-498 (2001).
81. Waters, J. S. et al. Phase I clinical and pharmacokinetic study of bcl-2 antisense oligonucleotide therapy in patients with non-Hodgkin's lymphoma. J Clin Oncol 18, 1812-1823 (2000).
82. Ramanarayanan, J., Hernandez-Ilizaliturri, F. J., Chanan-Khan, A. & Czuczman, M. S. Pro-apoptotic therapy with the oligonucleotide Genasense (oblimersen sodium) targeting Bd-2 protein expression enhances the biological anti-tumour activity of rituximab. Br J Haematol 127, 519-530 (2004).
83. Lee, Y. S. & Dutta, A. MicroRNAs in cancer. Annu Rev Pathol 4, 199-227 (2009).
84. He, L. & Hannon, G. J. MicroRNAs: small RNAs with a big role in gene regulation. Nat Rev Genet 5, 522-531 (2004).
85. Xiao, C. & Rajewsky, K. MicroRNA control in the immune system: basic principles. Cell 136, 26-36 (2009).
86. Sorrentino, A. et al. Role of microRNAs in drug-resistant ovarian cancer cells. Gynecol Oncol 111, 478-486 (2008).
87. Kovalchuk, O. et al. Involvement of microRNA-451 in resistance of the MCF-7 breast cancer cells to chemotherapeutic drug doxorubicin. Mol Cancer Ther 7, 2152-2159 (2008).
88. Zheng, T., Wang, J., Chen, X. & Liu, L Role of microRNA in anticancer drug resistance-int. Cancer 126, 2-10 (2010).
89. Xia, L et al. miR-15b and miR-16 modulate multidrug resistance by targeting BCL2 in human gastric cancer cells. Int J Cancer 123, 372-379 (2008).
90. Chen, J., Odenike, O. & Rowley, J. D. Leukaemogenesis: more than mutant genes. Natl Rev Cancer 10, 23-36 (2010).
91. Li, Z. et al. Up-regulation of a HOXA-PBX3 homeobox-gene signature following down-regulation of miR-181 is associated with adverse prognosis in patients with cytogenetically abnormal AML. Blood 119, 2314-2324 (2012).
92. Mi, S. et al. MicroRNA expression signatures accurately discriminate acute lymphoblastic leukemia from acute myeloid leukemia. Proc Natl Acad Sci USA 104, 19971-19976 (2007).
93. Arnold, C. P. et al. MicroRNA programs in normal and aberrant stem and progenitor cells. Genome Res 21, 798-810 (2011).
94. Schwind, S. et al. Prognostic significance of expression of a single microRNA, miR-181a, in cytogenetically normal acute myeloid leukemia: a Cancer and Leukemia Group B study. J Clin Oncol 28, 5257-5264 (2010).
95. Marcucci, G. et al. MicroRNA expression in cytogenetically normal acute myeloid leukemia. N Engl J Med 358, 1919-1928 (2008).
96. Marcucci, G. et al. Prognostic significance of, and gene and microRNA expression signatures associated with, CEBPA mutations in cytogenetically normal acute myeloid leukemia with high-risk molecular features: a Cancer and Leukemia Group B Study. J Clin Oncol 26, 5078-5087 (2008).
97. Lu, F. et al. miR-181b increases drug sensitivity in acute myeloid leukemia via targeting HMGB1 and Mcl-1. Int J Oncol 45, 383-392 (2014).
98. Nobumori, Y. et al. B56gamma tumor-associated mutations provide new mechanisms for B56gamma-PP2A tumor suppressor activity. Mol Cancer Res 11, 995-1003 (2013).
99. Yanez, A. G. Regulation of microRNA activity by translation initiation factors in melanoma (2014).
100. Bonness, K. et al. Cantharidin-induced mitotic arrest is associated with the formation of aberrant mitotic spindles and lagging chromosomes resulting, in part, from the suppression of PP2Aalpha. Mol Cancer Ther 5, 2727-2736 (2006).
101. Burgess, A. et al. Loss of human Greatwall results in G2 arrest and multiple mitotic defects due to deregulation of the cyclin B-Cdc2/PP2A balance. Proc Acad Sci USA 107, 12564-12569 (2010).
102. Zhao, Y. et al. Roles of Greatwall kinase in the regulation of cdc25 phosphatase. Mol Biol Cell 19, 1317-1327 (2008).
103. Vigneron, S. et al. Greatwall maintains mitosis through regulation of PP2A. EMBO J 28, 2786-2793 (2009).
104. Janssens, V. & Goris, J. Protein phosphatase 2A: a highly regulated family of serine/threonine phosphatases implicated in cell growth and signalling. Biochem 1353, 417-439 (2001).
105. Berry, L D. & Gould, K. L Regulation of Cdc2 activity by phosphorylation at T14/Y15. Prog Cell Cycle Res 2, 99-105 (1996).
106. Coleman, T. R. & Dunphy, W. G. Cdc2 regulatory factors. Curr Opin Cell Blot 6, 877-882 (1994).
107. Millar, J. et al. cdc25 M-phase inducer. Cold Spring Harb Symp Quant Biol 56, 577-584 (1991).
108. Yang, Y., Huang, Q., Lu, Y., Li, X. & Huang, S. Reactivating PP2A by FTY720 as a novel therapy for AML with C-KIT tyrosine kinase domain mutation. J Cell Biochem 113, 1314-1322 (2012).
109. Roberts, K. G. et al. Essential requirement for PP2A inhibition by the oncogenic receptor c-KIT suggests PP2A reactivation as a strategy to treat c-KIT+ cancers. Cancer Res 70, 5438-5447 (2010).
110. De Luca, A., Maiello, M. R., D'Akssio, A., Pergameno, M. & Normanno, N. The RAS/RAF/MEK/ERK and the PI3KJAKT signaling pathways: role in cancer pathogenesis and implications for therapeutic approaches. Expert Opin Ther Targets 16(Suppl 2), S17-27 (2012).
111. Ruvolo, P. P., Deng, X. & May, W. S. Phosphorylation of Bc12 and regulation of apoptosis. Leukemia 15, 515-522 (2001).
112. Ruvolo, P. P., Clark, W, Mumby, M., Gao, F. & May, W. S. A functional role for the B56 alpha-subunit of protein phosphatase 2A in ceramide-mediated regulation of Bc12 phosphorylation status and function. J Biol Chem 277, 22847-22852 (2002).

1. A method for treating secondary acute myeloid leukemia (sAML) in a patient comprising administering a PP2A inhibitor having the structure: 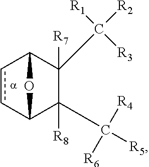
wherein:
bond α is present or absent;
R₁ and R₂ together are ═O;
R₃ is OH, O⁻, OR₉, O(CH₂)₁₋₆R₉, SH, S⁻, or SR₉, wherein R₉ is H, alkyl, alkenyl, alkynyl or aryl;
R₄ is 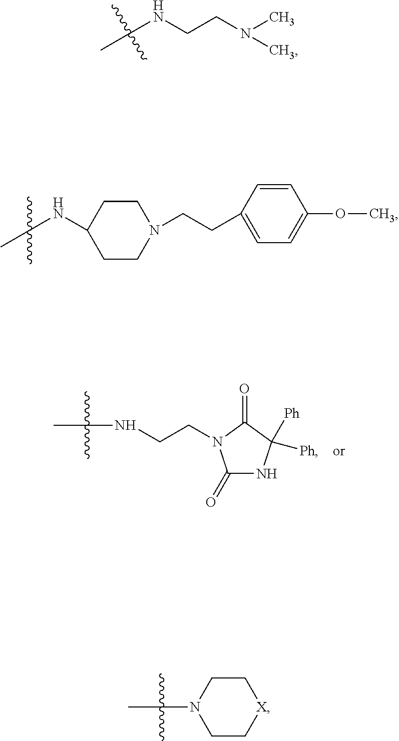
where X is O, S, NR₁₀, N⁺HR₁₀ or N⁺R₁₀R₁₀, where each R₁₀ is independently H, alkyl, alkenyl, alkynyl, aryl, 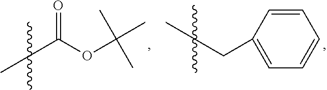
—CH₂CN, —CH₂CO₂R₁₁, or —CH₂COR₁₁, wherein each R₁₁ is independently H, alkyl, alkenyl or alkynyl;
R₅ and R₆ taken together are ═O;
R₇ and R₈ are each H,
or a salt, zwitterion, or ester thereof.

2. The method according to claim 1, wherein the PP2A inhibitor has the structure:

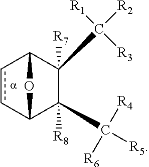

3. The method according to claim 1, wherein bond α is absent.

4. The method according to claim 1, wherein bond α is present.

5. The method according to claim 1, wherein the PP2A inhibitor has the structure: 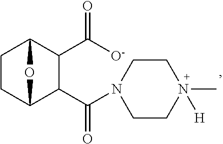
or a salt or ester thereof.

6. The method according to claim 1, further comprising administration of an anti-cancer agent.

7. The method according to claim 6, wherein the anti-cancer agent is daunorubicin.

8. The method according to claim 6, wherein the administration of the PP2A inhibitor enhances cytotoxicity of the anti-cancer agent.

9. The method according to claim 6, wherein the administration of the PP2A inhibitor enhances cytotoxicity of the anti-cancer agent via upregulation of miR-181b-1.

10. A method for treating secondary acute myeloid leukemia (sAML) in a patient comprising administering a PP2A inhibitor in combination with an anti-cancer agent so as to thereby treat sAML; wherein the PP2a inhibitor has the structure: 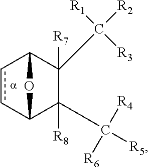
wherein:
bond α is present or absent;
R₁ and R₂ together are ═O;
R₃ is OH, O⁻, OR₉, O(CH₂)₁₋₆R₉, SH, S⁻, or SR₉, wherein R₉ is H, alkyl, alkenyl, alkynyl or aryl;
R₄ is 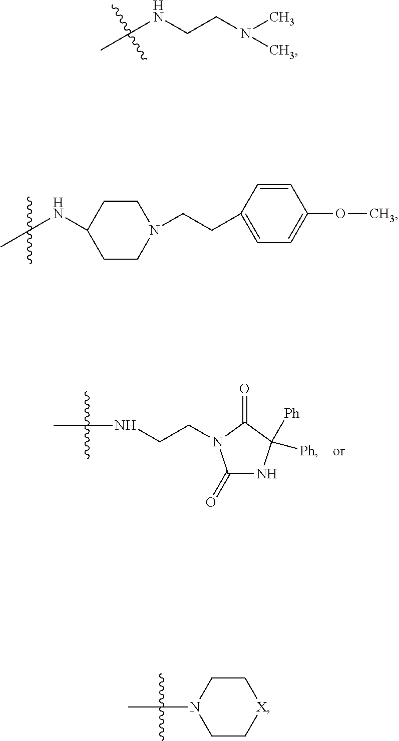
where X is O, S, NR₁₀, N⁺HR₁₀ or N⁺R₁₀R₁₀, where each R₁₀ is independently H, alkyl, alkenyl, alkynyl, aryl, 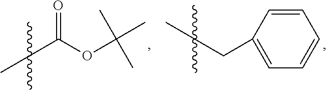
—CH₂CN, —CH₂CO₂R₁₁, or —CH₂COR₁₁, wherein each R₁₁ is independently H, alkyl, alkenyl or alkynyl;
R₅ and R₆ taken together are ═O;
R₇ and R₈ are each H,
or a salt, zwitterion, or ester thereof.

11. The method according to claim 10, wherein the PP2A inhibitor has the structure:

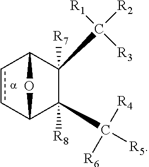

12-13. (canceled)

14. The method according to claim 10, wherein the PP2A inhibitor has the structure: 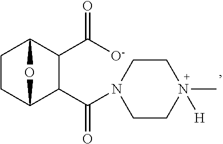
or a salt or ester thereof.

15. The method according to claim 10, wherein the anti-cancer agent is daunorubicin.

16. The method according to claim 10, wherein the administration of the PP2A inhibitor enhances cytotoxicity of the anti-cancer agent.

17. The method according to claim 10, wherein the administration of the PP2A inhibitor enhances cytotoxicity of the anti-cancer agent via upregulation of miR-181b-1.

18. The method according to claim 10, wherein the PP2A inhibitor and the anti-cancer agent are administered simultaneously, separately or sequentially.

19. A method of enhancing cytotoxicity of an anti-cancer agent in a patient afflicted with sAML comprising administering to the patient a PP2A inhibitor having the structure: 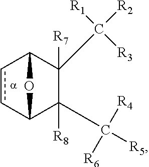
wherein:
bond α is present or absent;
R₁ and R₂ together are ═O;
R₃ is OH, O⁻, OR₉, O(CH₂)₁₋₆R₉, SH, S⁻, or SR₉, wherein R₉ is H, alkyl, alkenyl, alkynyl or aryl;
R₄ is 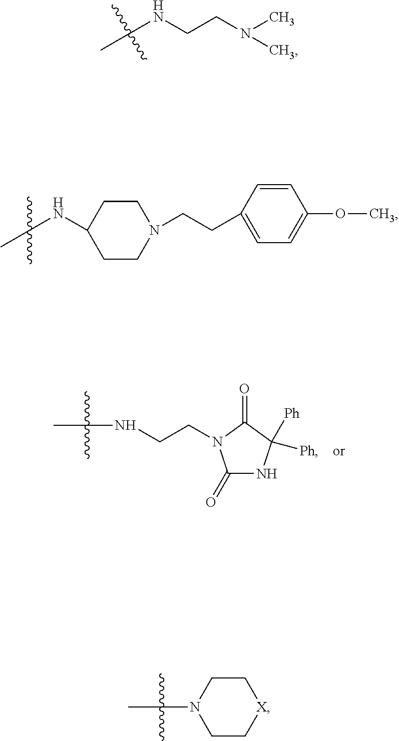
where X is O, S, NR₁₀, N⁺HR₁₀ or N⁺R₁₀R₁₀, where each R₁₀ is independently H, alkyl, alkenyl, alkynyl, aryl, 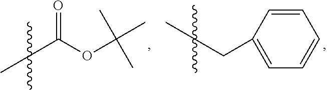
—CH₂CN, —CH₂CO₂R₁₁, or —CH₂COR₁₁, wherein each R₁₁ is independently H, alkyl, alkenyl or alkynyl;
R₅ and R₆ taken together are ═O;
R₇ and R₈ are each H,
or a salt, zwitterion, or ester thereof.

20-22. (canceled)

23. The method according to claim 19, wherein the PP2A inhibitor has the structure: 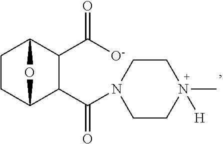
or a salt or ester thereof.

24. The method according to claim 19, wherein the anti-cancer agent is daunorubicin.

25. The method according to claim 19, wherein the administration of the PP2A inhibitor enhances cytotoxicity of the anti-cancer agent via upregulation of miR-181b-1.
